(19) United States
(12) Patent Application Publication
(10) Pub. No.: US 2021/0403511 A1
(43) Pub. Date: Dec. 30, 2021

(54) NUCLEIC ACIDS AND METHODS FOR GENOME EDITING

(71) Applicant: PILLARGO, INC., San Diego, CA (US)

(72) Inventors: Ying WU, San Diego, CA (US); Jiagang Zhao, San Diego, CA (US)

(21) Appl. No.: 16/475,275

(22) PCT Filed: Jan. 5, 2018
(86) PCT No.: PCT/US2018/012618

§ 371 Date: Jul. 1, 2019

Related U.S. Application Data
(60) Provisional application No. 62/443,515, filed on Jan. 6, 2017.

(51) Int. Cl. C07K 14/215; C12N 15/11; C12N 15/90

(52) U.S. Cl. CPC C07K 14/215; C12N 2310/20; C12N 15/907; C12N 15/11

(57) ABSTRACT

Presented herein, in certain embodiments, are nucleic acids and compositions for use in targeted gene editing.

### RELATED APPLICATIONS

This patent application claims the benefit of U.S. Provisional Patent Application No. 62/443,515 filed on Jan. 6, 2017, entitled NUCLEIC ACIDS AND METHODS FOR GENOME EDITING, naming Ying Wu and Jiagang Zhao as inventors, and designated by Attorney Docket No. 046432-0450783.

### FIELD OF THE INVENTION

Embodiments of the invention relate to compositions comprising synthetic nucleic acids that facilitate genome editing, and uses thereof.

### INTRODUCTION

Genome editing has offered a powerful tool and unprecedented opportunity to study gene functions and to fight diseases by introducing a targeted genomic sequence change at a specific locus of a living cell or organism. Zinc finger nucleases (ZFNs), transcription activator-like effector nucleases (TALENs) and clustered regularly interspaced short palindromic repeats (CRISPR)-associated (Cas) nucleases are the ones that have been used successfully and efficiently by many laboratories. More recently, the RNA-guided endonucleases such as Cas9 and Cpf1 have gained more tractions because of their relatively easiness on manipulation. The user-friendly CRISPR-Cas9 is very efficient in making mutations via nonhomologous end joining (NHEJ) in human cancer cell lines such as 293T cells. It also can mediate homologous recombination (HR), but at a much low efficiency (2-5%) in 293T cells and even lower in other biologically relevant cells such as human induced pluripotent stem cells (iPSCs).

Recently, a Chinese group reported that it is feasible to achieve genome editing by using Natronobacterium gregoryi Argonaute (NgAgo) with a guide DNA oligo in human cells (Gao F, et al., (2016) Nat Biotechnol. 34(7):768-73). However, multiple labs have failed to reproduce this phenomenon so far. Interestingly, an eye defect was observed by using an NgAgo approach in zebrafish (Qi J, et al., (2016) Cell Res. 26(12):1349-1352). However, the phenotype was most likely caused by an NgAgo mediated gene knockdown effect, and no genetic modification was observed at the DNA level.

Argonautes are a family of endonucleases that use 5′ phosphorylated short single-stranded nucleic acids as guides to cleave targets (Swarts D C, et al., (2014) Nat. Struct. Mol. Biol. 21(9):743-53 & Swarts D C, et al., (2014) Nature 507(7491):258-61). Similar to Cas9 and Cpf1, Argonautes play key roles in gene expression repression and host defense against foreign nucleic acids. While Cas9 and Cpf1 are only naturally found in prokaryotes, members of Argonaute superfamily are reported to be present in many species (from bacteria to mammals). Although most Argonautes associate with single-stranded (ss) RNAs and play a central role in RNA silencing, some Argonautes bind ssDNAs and cleave target DNAs (Swarts D C, et al., (2015) Nucleic Acids Res. 43(10):5120-9). It appears that DNA-guided Argonaute binding does not have a specific requirement for sequence or secondary structure. Argonautes are conserved through the bacterial and archaeal domains of living organisms. Their major functions are likely to be involved in DNA-guided DNA-interfering host defense systems.

Presented herein are novel modified Argonaute nucleic acids and uses thereof.

### SUMMARY OF THE INVENTION

In some aspects, presented herein is a nucleic acid comprising a first nucleic acid sequence having greater than 75% identity to the nucleic acid sequence of SEQ ID NO:1. In some aspects, presented herein is a nucleic acid comprising a first nucleic acid sequence having 75% to 100% identity to the nucleic acid sequence of SEQ ID NO:1.

In some aspects, presented herein is a nucleic acid comprising a first nucleic acid consisting of at least 30, at least 50, at least 75 or at least 100 contiguous nucleotides of the nucleic acid sequence of SEQ ID NO:1.

In some aspects, presented herein is a nucleic acid comprising a first nucleic acid sequence consisting of at least 40 nucleotides in length, wherein the first nucleic acid has at least 80% identity to a portion of the nucleic acid sequence of SEQ ID NO:1.

In some embodiments, a first nucleic acid is a synthetic nucleic acid. In certain embodiments, a first nucleic acid is not a naturally occurring nucleic acid.

In certain aspects, a nucleic acid described herein comprises a promoter, a leader sequence and/or a nuclear localization signal (NLS) sequence.

In certain aspects, presented herein is a composition comprising a first nucleic acid sequence having greater than 75% identity to the nucleic acid sequence of SEQ ID NO:1 and/or a first nucleic acid consisting of at least 30, at least 50, at least 75 or at least 100 contiguous nucleotides of the nucleic acid sequence of SEQ ID NO:1. In some embodiments, the composition is a pharmaceutical composition (e.g., a composition formulated for administration to a subject). In certain embodiments, a pharmaceutical composition comprises one or more of a pharmaceutical acceptable excipient, diluent, additive or carrier.

In some aspects, presented herein is a method of editing a genome of a cell, comprising, a) providing a cell or organism comprising a genome, b) contacting the cell or organism with (i) the synthetic nucleic acid of any one of claims 1 to 5, (ii) a guide oligonucleotide consisting of 18 to 30 nucleotides in length that is at least 90% identical to a target sequence in the genome and (iii) a donor sequence comprising a desired nucleic acid, a 5′-flanking sequence, and a 3′-flanking sequence, wherein each of the 5′-flanking sequence and the 3′-flanking sequence are located on opposite sides of the desired nucleic acid sequence and independently comprise at least 8 consecutive nucleotides that are identical to a portion of the guide sequence. In certain embodiments, the 5′-flanking sequence and the 3′-flanking sequence are 10 to 50 nucleotides in length. In some embodiments, the 5′-flanking sequence and the 3′-flanking sequence each comprise at least 10 nucleotides that are identical to the target sequence. In certain embodiments, the 5′ and the 3′ flanking sequences are different sequences. In some embodiments, the target sequence is 16 to 30 nucleotides in length. In some embodiments, the contacting step of the method comprises introducing the synthetic nucleic acid, the guide oligonucleotide and the donor sequence into the cell. The synthetic nucleic acid, the guide oligonucleotide and the donor sequence into the cell may be introduced into the cell using any suitable method (e.g., by transfection, transduction or electroporation, and the like). A cell may be a mammalian cell or a human cell. In certain embodiments, a desired nucleic acid comprises a human gene or portion thereof.

In some aspects, presented herein is a kit comprising one or more nucleic acids, and/or one or more compositions described herein.

### BRIEF DESCRIPTION OF THE FIGURES

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1A shows an NgAgo coding sequence and corresponding amino acid sequence of an NgAgo open reading frame. Underlined codons were considered for modification.

FIG. 1B shows a 2706 nucleotide modified Argonaute gene sequence (HuAgo).

FIG. 1C shows a sequence alignment between HuAgo and NgAgo. The start codon is shown in red. The nuclear localization signal (NLS) sequence is underlined.

FIG. 2 shows the last exon of a human COL8A2 gene (Top) illustrating the position of a 5′-targeted 20 bp sequence and a 3′ targeted 21 bp sequence (Top, underlined) for DNA-guided knock-in of an EGFP-P2A-Puromycin cassette (Middle), which is flanked by short homologous sequences (20 bp & 21 bp, as indicated by purple lines; Bottom) matching to both sides of the cleavage site in the last exon of COL8A2 gene. The COL8A2 genomic sequence is in capital letters and its stop codon TAA is in red. The 8-nt linker is in light brown; EGFP and puromycin ORFs are marked in green and blue respectively.

FIG. 3 shows a sequence representing a knock-in of a EGFP-P2A-Puro donor fragment into the human COL8A2 locus. The targeting oligonucleotides are underlined. COL8A2 genomic sequence is highlighted in yellow. Donor EGFP sequence is highlighted in green. The 8-nucleotide linker, which replaced the stop codon of COL8A2 gene and fused to EGFP open reading frame, is highlighted in gray.

FIG. 4 shows a representative hit of a BlastN search result.

FIG. 5 shows expression of GFP from the EGFP-Puro knock-in donor cassette targeted to the COL8A2 locus in HEK293T cells.

FIG. 6 shows a schematic representation of HuAgo full length and truncated constructs. All constructs contain the nuclear localization signal (NLS) at the N-terminal end.

FIGS. 7A and 7B. Point mutations were introduced to Exon 3 of the human CD274 gene by DNA oligo guided HuAgo in HEK293T cells. FIG. 7A shows the oligo guide GD5 target sequence is boldfaced. The top line is the reference sequence of the target region of the CD274 gene. Single point mutations of clonal amplicon #9 and #10 are underlined. FIG. 7B shows a chromatogram illustrating the A/G base change in amplicon #9 as indicated by the arrow.

### DETAILED DESCRIPTION

Presented herein, in some embodiments, are modified Argonaute nucleic acids, compositions comprising a modified Argonaute nucleic acid, kits and uses thereof.

The term “subject” refers to animals, typically mammalian animals. Any suitable mammal can be treated by a method or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In certain embodiments a mammal can be an animal disease model.

The terms “nucleic acid” refers to one or more nucleic acids (e.g., a set or subset of nucleic acids) of any composition from, such as DNA (e.g., complementary DNA (cDNA), genomic DNA (gDNA) and the like), RNA (e.g., message RNA (mRNA), short inhibitory RNA (siRNA), ribosomal RNA (rRNA), tRNA, microRNA, and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. In some embodiments a nucleic acid refers to DNA. In some embodiments a nucleic acid refers to RNA. Unless specifically limited, the term encompasses nucleic acids comprising deoxyribonucleotides, ribonucleotides and known analogs of natural nucleotides. A nucleic acid may include, as equivalents, derivatives, or variants thereof, suitable analogs of RNA or DNA synthesized from nucleotide analogs, single-stranded (“sense” or “antisense”, “plus” strand or “minus” strand, “forward” reading frame or “reverse” reading frame) and double-stranded polynucleotides. Nucleic acids may be single or double stranded. A nucleic acid can be of any length of 2 or more, 3 or more, 4 or more or 5 or more contiguous nucleotides. A nucleic acid can comprise a specific 5′ to 3′ order of nucleotides known in the art as a sequence (e.g., a nucleic acid sequence, e.g., a sequence).

A nucleic acid may be naturally occurring and/or may be synthesized, copied or altered (e.g., by a technician, scientist or one of skill in the art). For, example, a nucleic acid may be an amplicon. A nucleic acid may be from a nucleic acid library, such as a gDNA, cDNA or RNA library, for example. A nucleic acid can be synthesized (e.g., chemically synthesized) or generated (e.g., by polymerase extension in vitro, e.g., by amplification, e.g., by PCR). A nucleic acid may be, or may be from, a plasmid, phage, virus, autonomously replicating sequence (ARS), centromere, artificial chromosome, chromosome, or other nucleic acid able to replicate or be replicated in vitro or in a host cell, a cell, a cell nucleus or cytoplasm of a cell in certain embodiments. Nucleic acid provided for processes or methods described herein may comprise nucleic acids from 1 to 1000, 1 to 500, 1 to 200, 1 to 100, 1 to 50, 1 to 20 or 1 to 10 samples. Oligonucleotides are relatively short nucleic acids. Oligonucleotides can be from about 2 to 150, 2 to 100, 2 to 50, or 2 to about 35 nucleic acids in length. In certain embodiments, oligonucleotides are 18 to 30, 20 to 28 or 21-26 nucleotides in length. In some embodiments oligonucleotides are single stranded. In certain embodiments, oligonucleotides are primers. Primers are often configured to hybridize to a selected complementary nucleic acid and are configured to be extended by a polymerase after hybridizing.

A genome of a cell refers to the genetic material of a cell or organism. The genetic material of a cell or organism often comprises one or more genes. In certain embodiments a gene comprises or consists of one or more nucleic acids. The term “gene” means the segment of DNA involved in producing a polypeptide chain and can include coding regions (e.g., exons), regions preceding and following the coding region (leader and trailer) involved in the transcription/translation of the gene product and the regulation of the transcription/translation, as well as intervening sequences (introns) between individual coding segments (exons). A gene may not necessarily produce a peptide or may produce a truncated or non-functional protein due to genetic variation in a gene sequence (e.g., mutations in coding and non-coding portions of a gene). For example, a non-functional gene can be a pseudogene. A gene, whether functional or non-functional, can often be identified by homology to a gene in a reference genome. For example, any specific gene (e.g., a gene of interest, a counterpart gene, a pseudogene and the like) of a subject can be identified in another subject, genome or in a reference genome by one of skill in the art. In a diploid subject, a gene often comprises a pair of alleles (e.g., two alleles). Thus a method, system or process herein can be applied to one or both alleles of a gene. In some embodiments a method, system or process herein is applied to each allele of a gene.

The term “percent identical” or “percent identity” refers to sequence identity between two amino acid sequences. Identity can be determined by comparing a position in each sequence which may be aligned for purposes of comparison. When an equivalent position in the compared sequences is occupied by the same amino acid, then the molecules are identical at that position. When the equivalent site is occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. In some embodiments an alignment program that permits gaps in the sequence is utilized to align the sequences. The Smith-Waterman is one type of algorithm that permits gaps in sequence alignments. See Meth. Mol. Biol. 70:173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

In some embodiments a nucleic acid described herein comprises a label. As used herein, the terms “label” or “labeled” refers to incorporation of a detectable marker, e.g., by incorporation of a radiolabeled amino acid or attachment to a polypeptide of biotin moieties that can be detected by marked avidin (e.g., streptavidin containing a fluorescent marker or enzymatic activity that can be detected by optical or colorimetric methods). In certain embodiments, the label or marker can also be therapeutic. Various methods of labeling polypeptides and glycoproteins are known in the art and can be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹²⁵I, ¹³¹I), fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In certain embodiments, labels are attached by spacer arms of various lengths to reduce potential steric hindrance.

In some embodiments a carrier, radioisotope and/or a polypeptide can be indirectly or directly associated with, or bound to (e.g., covalently bound to, or conjugated to), a nucleic acid described herein. In certain embodiments agents or molecules are sometimes conjugated to or bound to nucleic acids to alter or extend the in vivo half-life of a nucleic acid or fragment thereof. In some embodiments, a nucleic acid described herein is fused or associated with one or more polypeptides (e.g., a toxin, ligand, receptor, cytokine, antibody, the like or combinations thereof). In certain embodiments, a nucleic acid described herein is linked to a half-life extending vehicle known in the art. Such vehicles include, but are not limited to, polyethylene glycol, glycogen (e.g., glycosylation of the antigen binding protein), and dextran. Such vehicles are described, e.g., in U.S. application Ser. No. 09/428,082, now U.S. Pat. No. 6,660,843 and published PCT Application No. WO 99/25044, hereby incorporated by reference.

In some embodiments carriers or anti-bacterial medications are bound to a nucleic acid described herein by a linker. A linker can provide a mechanism for covalently attaching a carrier and/or anti-bacterial medications to a nucleic acid described herein. Any suitable linker can be used in a composition or method described herein. Non-limiting examples of suitable linkers include silanes, thiols, phosphonic acid, and polyethylene glycol (PEG). Methods of attaching two or more molecules using a linker are well known in the art and are sometimes referred to as “crosslinking”. Non-limiting examples of crosslinking include an amine reacting with a N-Hydroxysuccinimide (NHS) ester, an imidoester, a pentafluorophenyl (PFP) ester, a hydroxymethyl phosphine, an oxirane or any other carbonyl compound; a carboxyl reacting with a carbodiimide; a sulfhydryl reacting with a maleimide, a haloacetyl, a pyridyldisulfide, and/or a vinyl sulfone; an aldehyde reacting with a hydrazine; any non-selective group reacting with diazirine and/or aryl azide; a hydroxyl reacting with isocyanate; a hydroxylamine reacting with a carbonyl compound; the like and combinations thereof.

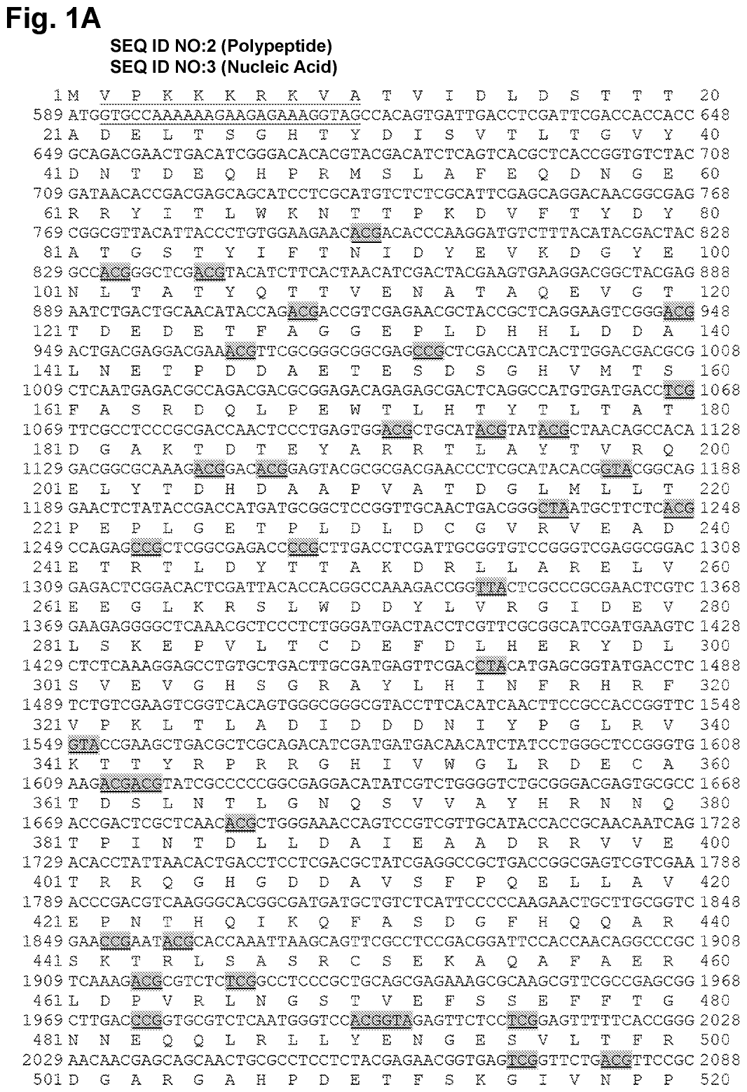

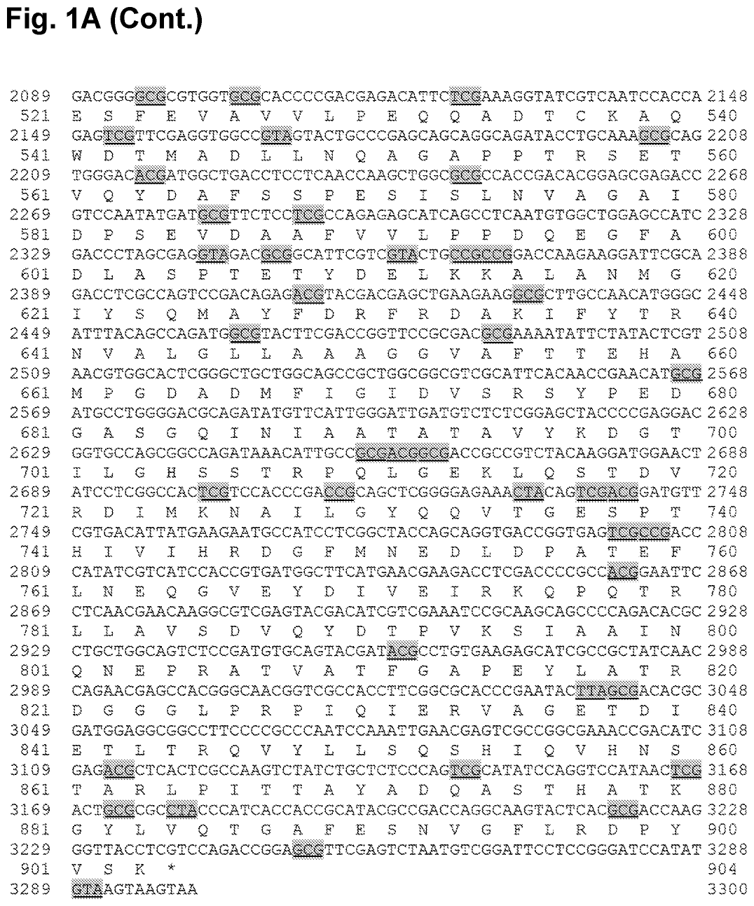

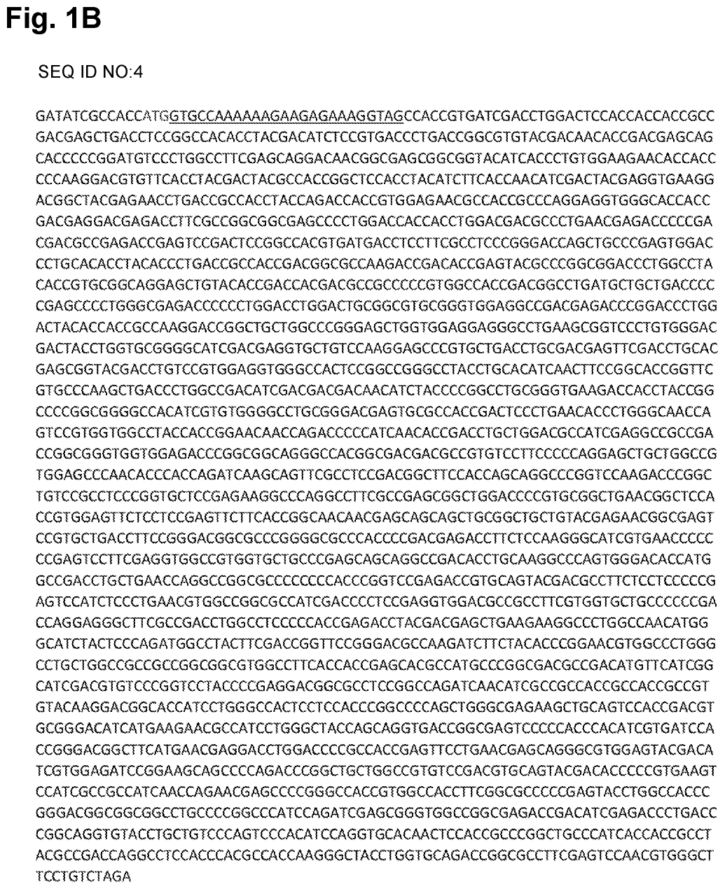

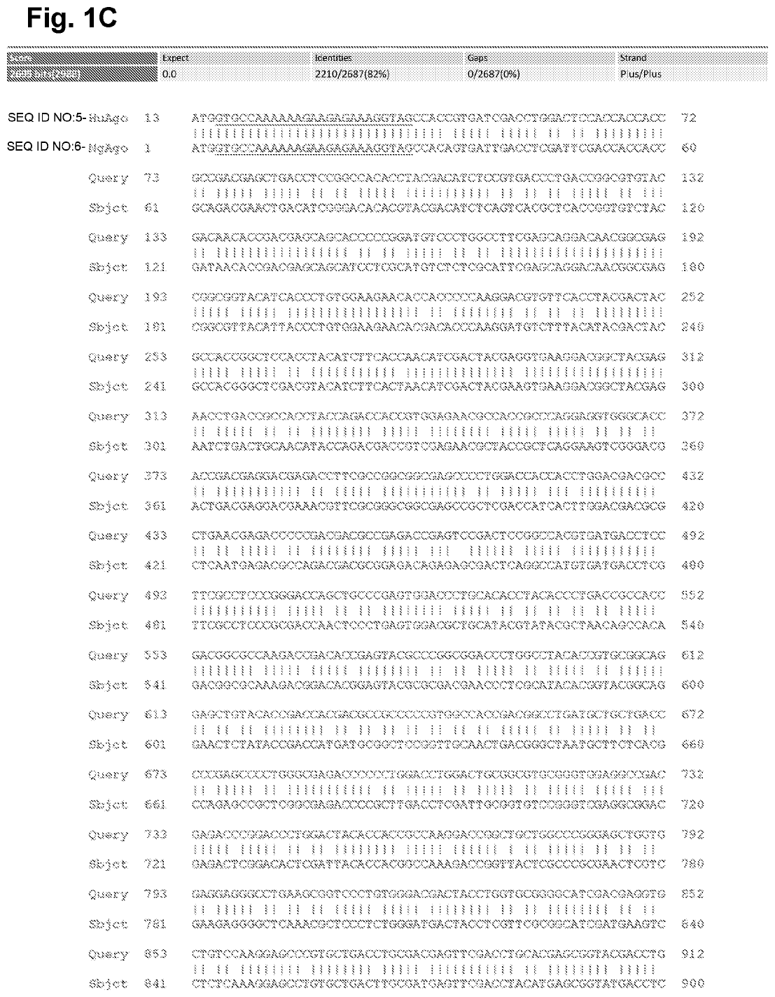

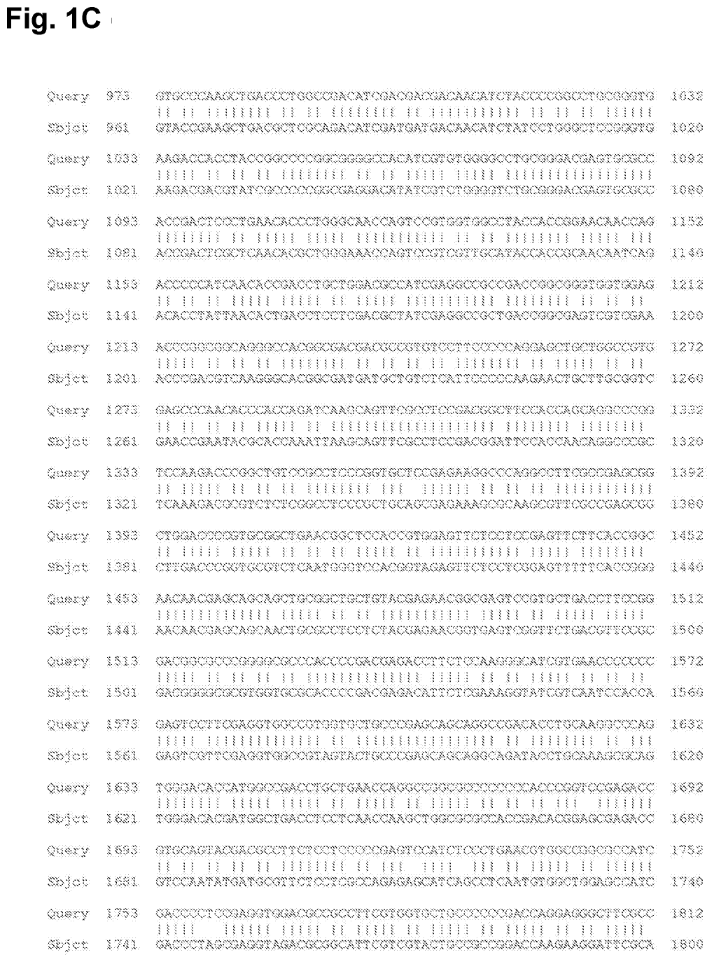

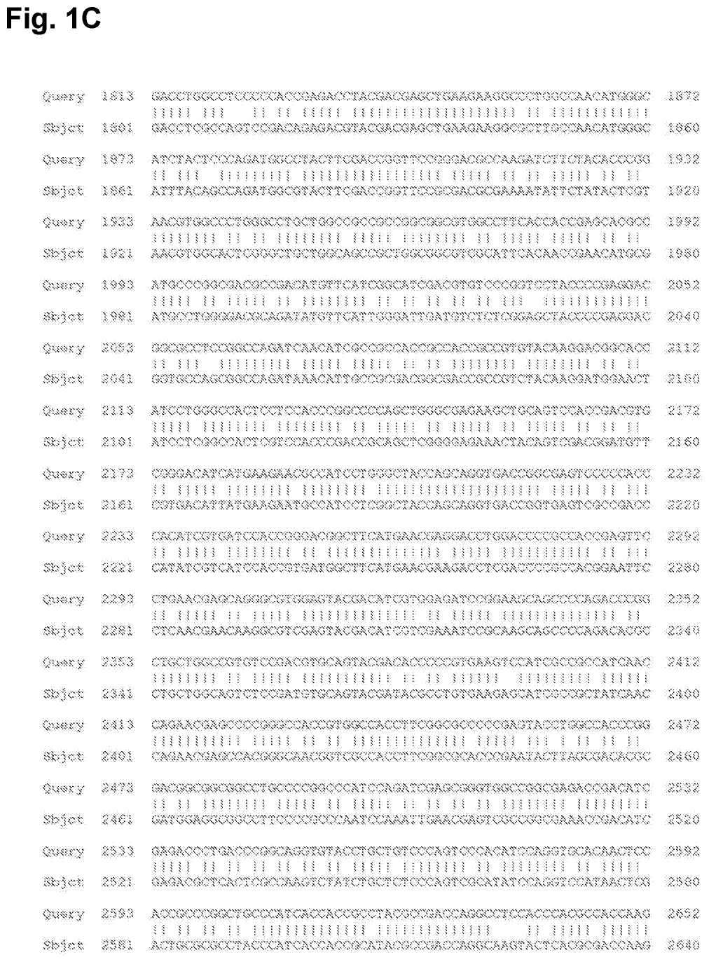

In certain embodiments, presented herein is a nucleic acid that encodes and/or expresses an Argonaute polypeptide or a functional fragment thereof. An Argonaute polypeptide is a DNA-guided endonuclease that can edit nucleic acids within a cell or subject (e.g., within a genome of a subject) in a target specific manner. In some embodiments, an Argonaute polypeptide comprises the polypeptide shown in FIG. 1A (SEQ ID NO:5). In certain embodiments, an Argonaute polypeptide comprises an amino acid sequence having 70% to 100 identity, 80% to 100% identity, 90% to 100% identity or 95% to 100% identity to the polypeptide shown in FIG. 1A (SEQ ID NO:5), or a portion thereof. In certain embodiments, an Argonaute polypeptide comprises an amino acid sequence having at least 75%, at least 80%, at least 85%, at least 90%, at least 95%, at least 98%, or at least 99% identity to the polypeptide sequence shown in FIG. 1A (SEQ ID NO:5), or a portion thereof. In some embodiments, an Argonaute polypeptide comprises a polypeptide encoded by the sequence of SEQ ID NO:1, SEQ ID NO:4 or SEQ ID NO:5. In certain embodiments, an Argonaute polypeptide comprises an amino acid sequence having 70% to 100 identity, 80% to 100% identity, 90% to 100% identity or 95% to 100% identity to a polypeptide encoded by the sequence of SEQ ID NO:1, SEQ ID NO:4 or SEQ ID NO:5, or a portion thereof. In certain embodiments, an Argonaute polypeptide comprises an amino acid sequence having at least 75%, at least 80%, at least 85%, at least 90%, at least 95%, at least 98%, or at least 99% identity to a polypeptide sequence encoded by the sequence of SEQ ID NO:1, SEQ ID NO:4 or SEQ ID NO:5, or a portion thereof. In certain embodiments, an Argonaute polypeptide or functional fragment thereof comprises and/or retains an ability to edit a target nucleic acid sequence (e.g., RNA, DNA, a gene, promoter or the like) within a cell (e.g., a cell of a subject) in a target specific manner. The ability to edit a target sequence within a genome of a subject refers to an ability to insert, remove and/or replace one or more specific nucleotides within a target sequence of a cell (e.g., a human cell). Accordingly, in certain embodiments, an Argonaute polypeptide, or functional fragment thereof is a polypeptide comprising an amino acid sequence having 70% to 100 identity, 80% to 100% identity, 90% to 100% identity or 95% to 100% identity to the polypeptide shown in FIG. 1A, or a portion thereof, wherein the Argo polypeptide and/or functional fragment thereof, comprises and/or retains an ability to edit a target sequence in a cell, in a target specific manner. In certain embodiments, an Argo polypeptide, or functional fragment thereof is a polypeptide comprising an amino acid sequence having at least 75%, at least 80%, at least 85%, at least 90%, at least 95%, at least 98%, or at least 99% identity to the polypeptide shown in FIG. 1A, or a portion thereof, wherein the Argo polypeptide and/or functional fragment thereof, comprises and/or retains an ability to edit a target sequence in a cell, in a target specific manner.

In certain embodiments, a functional fragment of an Argonaute polypeptide comprises a polypeptide sequence comprising at least 30, at least 50, at least 100, at least 200, at least 300, at least 400, at least 500, at least 600, at least 700 or at least 800 amino acids having 70% to 100 identity, 80% to 100% identity, 90% to 100% identity or 95% to 100% identity to the polypeptide shown in FIG. 1A. In certain embodiments, a functional fragment of an Argonaute polypeptide comprises a polypeptide sequence comprising at least 30, at least 50, at least 100, at least 200, at least 300, at least 400, at least 500, at least 600, at least 700 or at least 800 amino acids having at least 75%, at least 80%, at least 85%, at least 90%, at least 95%, at least 98%, or at least 99% identity to the polypeptide sequence shown in FIG. 1A.

In certain embodiments, an Argonaute polypeptide or functional fragment thereof comprises a polypeptide encoded by portion of a nucleotide sequence of SEQ ID NO:1, SEQ ID NO:4 or SEQ ID NO:5, wherein the encoded polypeptide, when expressed in a cell, comprises and/or retains an ability to edit a target nucleic acid sequence within the cell. In certain embodiments, an Argonaute polypeptide or functional fragment thereof comprises a polypeptide encoded by portion of the nucleotide sequence of SEQ ID NO:1, SEQ ID NO:4 or SEQ ID NO:5, wherein the encoded polypeptide, when expressed in a cell, comprises and/or retains an ability to edit a target nucleic acid sequence (target site) within the cell. In some embodiments, an Argonaute polypeptide or functional fragment thereof comprises a polypeptide encoded by a portion of a nucleotide sequence shown in FIG. 1A or FIG. 1B, wherein the encoded polypeptide, when expressed in a cell, comprises and/or retains DNA-guided endonuclease activity. In some embodiments, an Argonaute polypeptide or functional fragment thereof comprises a polypeptide encoded by portion of the nucleotide sequence of SEQ ID NO:1, SEQ ID NO:4 or SEQ ID NO:5, wherein the encoded polypeptide, when expressed in a cell, comprises and/or retains DNA-guided endonuclease activity.

In some embodiments, a nucleic acid herein encodes all or a portion of an Argonaute polypeptide, non-limiting examples of which include a portion that is 1 to 903 amino acids in length, or at least 50, at least 100, at least 200, at least 300, or at least 500 amino acids in length. In some embodiments, a nucleic acid herein encodes all or a portion of the Argonaute polypeptide shown in FIG. 1A, wherein the Argonaute polypeptide, or portion thereof (e.g., a functional portion thereof; e.g., a functional fragment thereof), comprises nuclease activity and/or retains the ability to insert a heterologous nucleic acid sequence into the genome of a living mammalian cell at a specific targeted locus. In some embodiments, a nucleic acid that encodes an Argonaute polypeptide, or portion thereof (e.g., a functional portion thereof), is 80% to 100% identical to the Argonaute nucleic acid shown in FIG. 1A or FIG. 1B, or at least 80%, at least 81%, at least 82%, at least 85%, at least 90% or at least 95% identical to the Argonaute nucleic acid sequence of FIG. 1A or FIG. 1B.

In some embodiments, a nucleic acid (i.e., an Argonaute nucleic acid sequence) that encodes all or a portion of an Argonaute polypeptide (e.g., a functional Argonaute polypeptide) is a nucleic acid sequence having 80% to 100% identity to the nucleic acid sequence of SEQ ID NO:1. In certain embodiments, a nucleic acid (e.g., an Argonaute nucleic acid sequence) is at least 80%, at least 81%, at least 82%, at least 85%, at least 90% or at least 95% identical to the nucleic acid sequence of SEQ ID NO:1. A nucleic acid described herein if often not a naturally occurring nucleic acid and is often not found in nature. In certain embodiments, a nucleic acid described herein is a synthetic nucleic acid. A synthetic nucleic acid refers to a nucleic acid sequence that is designed by the hand of man and is not found in nature.

In some embodiments, a nucleic acid that encodes all or a portion an Argonaute polypeptide (i.e., an Argonaute nucleic acid) is a nucleic acid that comprises or consists of 50 to 2666 contiguous nucleotides (nt) of the nucleic acid sequence of SEQ ID NO:1. In certain embodiments, a nucleic acid comprises or consists of at least 50, at least 100, at least 500, at least 750, at least 1000, at least 1500, at least 1750 or at least 2000 contiguous nucleotides of the nucleic acid sequence of SEQ ID NO:1. In some embodiments, a nucleic acid or synthetic nucleic acid described herein consists of, or comprises, the nucleic acid sequence of SEQ ID NO:1.

In certain embodiments, a nucleic acid or synthetic nucleic acid described herein comprises a nucleic acid sequence that is 100 to 3000 nucleotides in length having 80% to 100% identity to the nucleic acid sequence of SEQ ID NO:1. In certain embodiments, a nucleic acid or synthetic nucleic acid described herein comprises a first nucleic acid sequence that is at least 100, at least 500, at least 750, at least 1000, at least 1500, at least 1750 or at least 2000 nucleotides in length, where the first nucleic acid has at least 80%, at least 81%, at least 82%, at least 85%, at least 90% or at least 95% identity to the nucleic acid sequence of SEQ ID NO:1.

In certain embodiments, a nucleic acid is configured to express a polypeptide in a mammalian cell. A nucleic acid that is configured to express a polypeptide (e.g., an Argonaute polypeptide) comprises one or more nucleic acid regulatory sequences that direct the expression of a polypeptide in a cell. Accordingly, a nucleic acid that is configured to express a desired polypeptide may include one or more of a coding region that encodes the desired protein, one or more suitable promoters operably linked to the coding region, a translation initiation sequence, a start codon, a stop codon, a polyA signal sequence, a leader sequence, a nuclear localization sequence, and the like. In certain embodiments a nucleic acid comprises a sequence that encodes a nuclear localization signal (NLS) sequence. Any suitable NLS sequence can be used. One non-limiting example of an NLS sequence is shown underlined in FIG. 1A. In certain embodiments, a nucleic acid is configured to express an Argonaute polypeptide, or functional fragment thereof.

In certain embodiments, a nucleic acid is a guide oligonucleotide (oligonucleotide guide). In some embodiments, a guide oligonucleotide comprises RNA. In certain embodiments, a guide oligonucleotide comprises DNA. In some embodiments, a guide oligonucleotide is 18 to 30, 18 to 28, 18 to 25, 18 to 26, 19 to 26, 19 to 25, 20 to 30, 20 to 25, 20 to 24 or 21 to 24 nucleotides in length. A guide oligonucleotide is sometimes a nucleic acid that is 18 to 30 nt in length. In certain embodiments, a guide oligonucleotide is 18, 19, 20, 21, 22, 23, 24, 25, 26, 27, 28, 29 or 30 nucleotides in length. A guide oligonucleotide often comprises a nucleic acid sequence that is 80% to 100%. 85% to 100%, 90% to 100%, 95% to 100%, or 100% identical to a target nucleic acid sequence (target site), or a portion thereof, located at a specific location within the genome of an organism or cell. In some embodiments, a guide oligonucleotide is 100% identical to a portion of a target sequence that is 18 to 30, 18 to 28, 18 to 25, 18 to 26, 19 to 26, 19 to 25, 20 to 30, 20 to 25, 20 to 24 or 21 to 24 nucleotides in length. In some embodiments, a guide oligonucleotide is 100% identical to a portion of a gene, intron or exon that is 18 to 30, 18 to 28, 18 to 25, 18 to 26, 19 to 26, 19 to 25, 20 to 30, 20 to 25, 20 to 24 or 21 to 24 nucleotides in length.

A target sequence refers to a specific location (a specific nucleic acid sequence) within the genome of an organism or cell that one intends to modify using a composition or method described herein. In some embodiments, a target sequence is a nucleic acid located within a genome of a cell or organism. In some embodiments, a target sequence comprises RNA. In certain embodiments, a target sequence comprises DNA. In certain embodiments, a target sequence is 10 to 100, 18 to 50, 18 to 30, 18 to 28, 18 to 25, 18 to 26, 19 to 26, 19 to 25, 20 to 30, 20 to 25, 20 to 24 or 21 to 24 nucleotides in length and may be located within a gene, exon, intron or any suitable portion of a genome. Any nucleotide within a target sequence or any portion of a target sequence can be modified by a method described herein. Any number of nucleotides within a target sequence may be deleted, mutated or replaced, for example by a desired sequence (e.g., an insert sequence of a donor sequence). In some embodiments, one or more nucleotides or a desired sequence are inserted into a target sequence by a method described herein. In certain embodiments a target sequence provides a nucleic acid sequence that is complementary or identical to a guide oligonucleotide, or portion thereof. In certain embodiments a target sequence provides a nucleic acid sequence that is complementary or identical to a 5′ and/or 3′ flanking regions of a donor sequence. For example, a guide oligonucleotide of 18-28 nucleotides in length may be 85% to 100%, 90% to 100%, or 95% to 100% identical to a target sequence. In certain embodiments, a guide oligonucleotide comprises 1 to 2, 1 to 3, or 1, 2, 3 or 4 mismatches compared to a target sequence in the genome. In some embodiments, a guide oligonucleotide is 100% identical to a target sequence. In some embodiments, a guide oligonucleotide comprises or consists of a sequence that is an exact match to a target site in a genome (i.e., a target sequence). In certain embodiments, a guide oligonucleotide is phosphorylated. In some embodiments, a guide oligonucleotide is 5′ phosphorylated (i.e., phosphorylated on the 5′-hydroxyl terminus).

Without being limited to theory, a functional Argonaute polypeptide described herein utilizes a guide oligonucleotide to cut the genomic DNA of an organism or cell at a specific target sequence that is defined by the sequence of a guide oligonucleotide. In certain embodiments, an Argonaute polypeptide cleaves a target nucleic acid sequence anywhere within a sequence defined by a guide oligonucleotide. In some embodiments, an Argonaute polypeptide cleaves a target site at a location defined by any one of the first 10 nucleotides (5′-nucleotides) of a guide oligonucleotide. When both a guide oligonucleotide and a donor nucleic acid are present, an Argonaute polypeptide will proceed to insert and splice a donor nucleic acid into the genome of a cell at a target sequence site defined by the guide oligonucleotide. If a donor sequence is not present, an Argonaute polypeptide loaded with a guide oligonucleotide, will often cleave a target site defined by the guide oligonucleotide sequence. This process often results in the introduction of one or more single nucleotide mutations introduced at the target site (e.g., see Example 3).

A donor sequence (a donor fragment) is a nucleic acid comprising three parts, a 5′ flanking sequence, a desired sequence and a 3′ flanking sequence. In some embodiments, a donor sequence comprises RNA. In certain embodiments, a donor sequence comprises DNA. In some embodiments, a donor sequences is single stranded. In some embodiments, a donor sequence is double stranded.

The 5′-flanking sequence and the 3′-flanking sequences may be the same sequence or different sequences. In some embodiments, the 5′-flanking sequence and the 3′-flanking sequences are different sequences that do not share more than 10% identity. In some embodiments, the 5′-flanking sequence and the 3′-flanking sequences are located on opposite sides of the desired sequence. A 5′ flanking sequence and/or a 3′ flanking sequence of a donor sequence, in certain embodiments, is about 10-100, 10-50, 10-75, 10-25 or 20-25 nucleotides (nt) or base pairs (bp) in length.

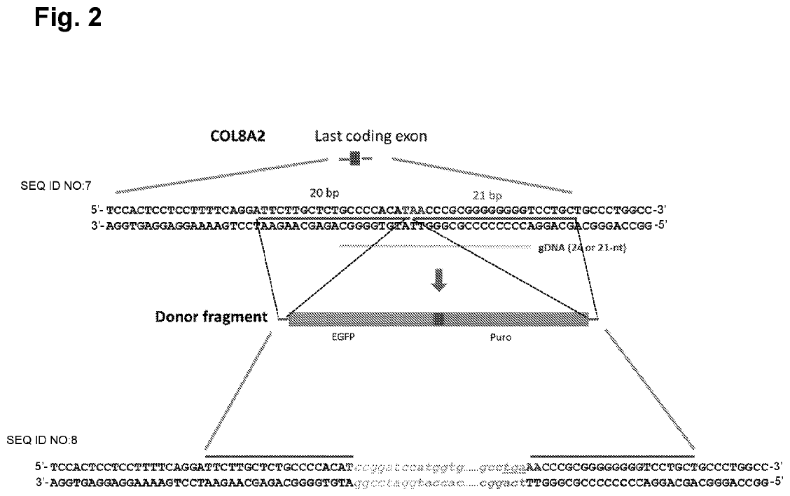

A desired sequence refers to a nucleic acid that is to be inserted into a target sequence by an Argonaute polypeptide and/or by a method described herein. The term “desired sequence” is used synonymously with the terms “desired nucleic acid” and “desired nucleic acid sequence”. For purposes of clarity, a “desired sequence” may sometimes be referred to as an “insert sequence”. In some embodiments, a desired sequence comprises RNA. In certain embodiments, a desired sequence comprises DNA. A desired sequence can be any suitable sequence of any suitable length. In some embodiments, a desired sequence is 1-20,000, 1-5,000, 1-1000, 1-500, 10-5,000, 10-1000 or 10-500 nucleotides long. In some embodiments, a donor sequence comprises a 5′ flanking sequence and a 3′ flanking sequence that are each, independently, 80% to 100% identical to a target site, which flanking sequences also comprise a sequence region that is identical to or complementary to a portion of a guide oligonucleotide (e.g., see FIG. 2 example). In certain embodiments, a donor sequence comprises a sequence that is 80% to 100% identical to a guide oligonucleotide and/or 80% to 100% identical to a target site. A donor sequence may comprise flanking sequences that are identical to a guide oligonucleotide or that overlap with a portion of a guide oligonucleotide. In certain embodiments, a donor sequence comprises (i) a desired sequence, (ii) a 5′ flanking sequence that is at least 80% to 100% identical to a guide oligonucleotide, or portion thereof, and/or 80% to 100% identical to a target site, and a 3′ flanking sequence that is at least 80% to 100% identical to a guide oligonucleotide, or portion thereof, and/or 80% to 100% identical to a target site. In certain embodiments, a guide oligonucleotide and a functional Argonaute polypeptide are sufficient to insert a desired sequence into a target site of a genome where a donor nucleic acid (i.e., a donor sequence, a donor nucleic acid sequence) comprises the desired sequence which is flanked on both sides (5′ side and 3′ side) by a sequence that is identical to a portion (e.g., at least 5 to 10 nucleotides) of the guide oligonucleotide. In certain embodiments, two guide oligonucleotides are used to insert a desired nucleic acid into a target site of a genome where a donor nucleic acid (donor sequence) comprises the desired sequence flanked on one side by a sequence of a first guide oligonucleotide and on the other side by a sequence of a second guide oligonucleotide.

According, in certain embodiments, presented herein is a method of editing a genome of an organism or cell. In certain embodiments, the organism is a subject. In some embodiments, the subject is a human. A cell may be any suitable cell, non-limiting examples of which include a prokaryotic cell, plant cell, eukaryotic cell, mammalian cell or human cell. In certain embodiments, a method of editing a genome comprises removal of a target sequence from a genome, disruption of a target sequence within a genome and/or insertion of a desired sequence into a genome. A desired sequence can be any suitable nucleic acid sequence non-limiting examples of which include a heterologous nucleic acid (e.g., from a different species), a modified heterologous nucleic acid, a homologous nucleic acid (e.g., from the same species), a synthetic nucleic acid, a gene or portion thereof (e.g., intron, exon, regulatory sequences, etc.), a modified gene, a marker, a toxin, a single nucleic acid, two or more nucleic acids, the like or combinations thereof. In some embodiments, a desired nucleic acid encodes a chimeric antigen receptor (CAR).

A desired nucleic acid (desired sequence) or gene can be any suitable mammalian gene, portion thereof, or modified form thereof, non-limiting examples of which include human genes A2M, AACS, AARSD1, ABCA10, ABCA12, ABCA3, ABCA8, ABCA9, ABCB1, ABCB10, ABCB4, ABCC11, ABCC12, ABCC6, ABCD1, ABCE1, ABCF1, ABCF2, ABT1, ACAA2, ACCSL, ACER2, ACO2, ACOT1, ACOT4, ACOT7, ACP1, ACR, ACRC, ACSBG2, ACSM1, ACSM2A, ACSM2B, ACSM4, ACSM5, ACTA1, ACTA2, ACTB, ACTG1, ACTG2, ACTN1, ACTN4, ACTR1A, ACTR2, ACTR3, ACTR3C, ACTRT1, ADAD1, ADAL, ADAM18, ADAM20, ADAM21, ADAM32, ADAMTS7, ADAMTSL2, ADAT2, ADCY5, ADCY6, ADCY7, ADGB, ADH1A, ADH1B, ADH1C, ADH5, ADORA2B, ADRBK2, ADSS, AFF3, AFF4, AFG3L2, AGAP1, AGAP10, AGAP11, AGAP4, AGAP5, AGAPE, AGAP7, AGAPE, AGAP9, AGER, AGGF1, AGK, AGPAT1, AGPAT6, AHCTF1, AHCY, AHNAK2, AHRR, AIDA, AIF1, AIM1L, AIMP2, AK2, AK3, AK4, AKAP13, AKAP17A, AKIP1, AKIRIN1, AKIRIN2, AKR1B1, AKR1B10, AKR1B15, AKR1C1, AKR1C2, AKR1C3, AKR1C4, AKR7A2, AKR7A3, AKTIP, ALDH3B1, ALDH3B2, ALDH7A1, ALDOA, ALG1, ALG10, ALG10B, ALG1L, ALG1L2, ALG3, ALKBH8, ALMS1, ALOX15, ALOX15B, ALOXE3, ALPI, ALPP, ALPPL2, ALYREF, AMD1, AMELX, AMELY, AMMECR1L, AMY1A, AMY1B, AMY1C, AMY2A, AMY2B, AMZ2, ANAPC1, ANAPC10, ANAPC15, ANKRD11, ANKRD18A, ANKRD18B, ANKRD20A1, ANKRD20A19P, ANKRD20A2, ANKRD20A3, ANKRD20A4, ANKRD30A, ANKRD30B, ANKRD36, ANKRD36B, ANKRD49, ANKS1B, ANO10, ANP32A, ANP32B, ANXA2, ANXA2R, ANXA8, ANXA8L1, ANXA8L2, AOC2, AOC3, AP1B1, AP1S2, AP2A1, AP2A2, AP2B1, AP2S1, AP3M2, AP3S1, AP4S1, APBA2, APBB1IP, APH1B, API5, APIP, APOBEC3A, APOBEC3B, APOBEC3C, APOBEC3D, APOBEC3F, APOBEC3G, APOC1, APOL1, APOL2, APOL4, APOM, APOOL, AQP10, AQP12A, AQP12B, AQP7, AREG, AREGB, ARF1, ARF4, ARF6, ARGFX, ARHGAP11A, ARHGAP11B, ARHGAP20, ARHGAP21, ARHGAP23, ARHGAP27, ARHGAP42, ARHGAP5, ARHGAP8, ARHGEF35, ARHGEF5, ARID2, ARID3B, ARIH2, ARL14EP, ARL16, ARL17A, ARL17B, ARL2BP, ARL4A, ARL5A, ARL6IP1, ARL6IP6, ARL8B, ARMC1, ARMC10, ARMC4, ARMC8, ARMCX6, ARPC1A, ARPC2, ARPC3, ARPP19, ARSD, ARSE, ARSF, ART3, ASAH2, ASAH2B, ASB9, ASL, ASMT, ASMTL, ASNS, ASS1, ATAD1, ATAD3A, ATAD3B, ATAD3C, ATAT1, ATF4, ATF6B, ATF7IP2, ATG4A, ATM ATMIN, ATP13A4, ATP13A5, ATP1A2, ATP1A4, ATP1B1, ATP1B3, ATP2B2, ATP2B3, ATP5A1, ATP5C1, ATP5F1, ATP5G1, ATP5G2, ATP5G3, ATP5H, ATP5J, ATP5J2, ATP5J2-PTCD1, ATP5O, ATP6AP2, ATP6V0C, ATP6V1E1, ATP6V1F, ATP6V1G1, ATP6V1G2, ATP7B, ATP8A2, ATP9B, ATXN1L, ATXN2L, ATXN7L3, AURKA, AURKAIP1, AVP, AZGP1, AZI2, B3GALNT1, B3GALT4, B3GAT3, B3GNT2, BAG4, BAG6, BAGE2, BAK1, BANF1, BANP, BCAP31, BCAR1, BCAS2, BCL2A1, BCL2L12, BCL2L2-PABPN1, BCLAF1, BCOR, BCR, BDH2, BDP1, BEND3, BET1, BEX1, BHLHB9, BHLHE22, BHLHE23, BHMT, BHMT2, BIN2, BIRC2, BIRC3, BLOC1S6, BLZFl, BMP2K, BMP8A, BMP8B, BMPR1A, BMS1, BNIP3, BOD1, BOD1L2, BOLA2, BOLA2B, BOLA3, BOP1, BPTF, BPY2, BPY2B, BPY2C, BRAF, BRCA1, BRCC3, BRD2, BRD7, BRDT, BRI3, BRK1, BRPF1, BRPF3, BRWD1, BTBD10, BTBD6, BTBD7, BTF3, BTF3L4, BTG1, BTN2A1, BTN2A2, BTN3A1, BTN3A2, BTN3A3, BTNL2, BTNL3, BTNL8, BUB3, BZW1, C10orf129, C10orf88, C11orf48, C11orf58, C11orf74, C11 orf75, C12orf29, C12orf42, C12orf49, C12orf71, C12orf76, C14orf119, C14orf166, C14orf178, C15orf39, C15orf40, C15orf43, C16orf52, C16orf88, C17orf51, C17orf58, C17orf61, C17orf89, C17orf98, C18orf21, C18orf25, C1D, C1GALT1, C1QBP, C1QL1, C1QL4, C1QTNF9, C1QTNF9B, C1QTNF9B-AS1, C1orf100, C1orf106, C1orf114, C2, C22orf42, C22orf43, C2CD4A, C2orf16, C2orf27A, C2orf27B, C2orf69, C2orf78, C2orf81, C4A, C4B, C4BPA, C4orf27, C4orf34, C4orf46, C5orf15, C5orf43, C5orf52, C5orf60, C5orf63, C6orf10, C6orf106, C6orf136, C6orf15, C6orf203, C6orf25, C6orf47, C6orf48, C7orf63, C7orf73, C8orf46, C9orf123, C9orf129, C9orf172, C9orf57, C9orf69, C9orf78, CA14, CA15P3, CA5A, CA5B, CABYR, CACNA1C, CACNA1G, CACNA1H, CACNA1I, CACYBP, CALCA, CALCB, CALM1, CALM2, CAMSAP1, CAP1, CAPN8, CAPZA1, CAPZA2, CARD16, CARD17, CASC4, CASP1, CASP3, CASP4, CASP5, CATSPER2, CBR1, CBR3, CBWD1, CBWD2, CBWD3, CBWD5, CBWD6, CBWD7, CBX1, CBX3, CCDC101, CCDC111, CCDC121, CCDC127, CCDC14, CCDC144A, CCDC144NL, CCDC146, CCDC150, CCDC174, CCDC25, CCDC58, CCDC7, CCDC74A, CCDC74B, CCDC75, CCDC86, CCHCR1, CCL15, CCL23, CCL3, CCL3L1, CCL3L3, CCL4, CCL4L1, CCL4L2, CCNB1IP1, CCNB2, CCND2, CCNG1, CCNJ, CCNT2, CCNYL1, CCR2, CCR5, CCRL1, CCRN4L, CCT4, CCT5, CCT6A, CCT7, CCT8, CCT8L2, CCZ1, CCZ1B, CD177, CD1A, CD1B, CD1C, CD1D, CD1E, CD200R1, CD200R1L, CD209, CD276, CD2BP2, CD300A, CD300C, CD300LD, CD300LF, CD33, CD46, CD83, CD8B, CD97, CD99, CDC14B, CDC20, CDC26, CDC27, CDC37, CDC42, CDC42EP3, CDCA4, CDCA7L, CDH12, CDK11A, CDK11B, CDK2AP2, CDK5RAP3, CDK7, CDK8, CDKN2A, CDKN2AIPNL, CDKN2B, CDON, CDPF1, CDRT1, CDRT15, CDRT15L2, CDSN, CDV3, CDY1, CDY2A, CDY2B, CEACAM1, CEACA11118, CEACAM21, CEACAM3, CEACAM4, CEACAM5, CEACAM6, CEACAM7, CEACAM8, CEL, CELA2A, CELA2B, CELA3A, CELA3B, CELSR1, CEND1, CENPC1, CENPI, CENPJ, CENPO, CEP170, CEP19, CEP192, CEP290, CEP57L1, CES1, CES2, CES5A, CFB, CFC1, CFC1B, CFH, CFHR1, CFHR2, CFHR3, CFHR4, CFHR5, CFL1, CFTR, CGB, CGB1, CGB2, CGB5, CGB7, CGB8, CHAF1B, CHCHD10, CHCHD2, CHCHD3, CHCHD4, CHD2, CHEK2, CHIA, CHMP4B, CHMP5, CHORDC1, CHP1, CHRAC1, CHRFAM7A, CHRNA2, CHRNA4, CHRNB2, CHRNB4, CHRNE, CHST5, CHST6, CHSY1, CHTF8, CIAPIN1, CIC, CIDEC, CIR1, CISD1, CISD2, CKAP2, CKMT1A, CKMT1B, CKS2, CLC, CLCN3, CLCNKA, CLCNKB, CLDN22, CLDN24, CLDN3, CLDN4, CLDN6, CLDN7, CLEC17A, CLEC18A, CLEC18B, CLEC18C, CLEC1A, CLEC1B, CLEC4G, CLEC4M, CLIC1, CLIC4, CLK2, CLK3, CLK4, CLNS1A, CMPK1, CMYA5, CNEP1R1, CNN2, CNN3, CNNM3, CNNM4, CNOT6L, CNOT7, CNTNAP3, CNTNAP3B, CNTNAP4, COA5, COBL, COIL, COL11A2, COL12A1, COL19A1, COL25A1, COL28A1, COL4A5, COL6A5, COL6A6, COWD4, COWD5, COPRS, COPS5, COPS8, COQ10B, CORO1A, COX10, COX17, COX20, COX5A, COX6A1, COX6B1, COX7B, COX7C, COX8C, CP, CPAMD8, CPD, CPEB1, CPSF6, CR1, CR1L, CRADD, CRB3, CRCP, CREBBP, CRHR1, CRLF2, CRLF3, CRNN, CROCC, CRTC1, CRYBB2, CRYGB, CRYGC, CRYGD, CS, CSAG1, CSAG2, CSAG3, CSDA, CSDE1, CSF2RA, CSF2RB, CSGALNACT2, CSH1, CSH2, CSHL1, CSNK1A1, CSNK1D, CSNK1E, CSNK1G2, CSNK2A1, CSNK2B, CSPG4, CSRP2, CST1, CST2, CST3, CST4, CST5, CST9, CT45A1, CT45A2, CT45A3, CT45A4, CT45A5, CT45A6, CT47A1, CT47A10, CT47A11, CT47A12, CT47A2, CT47A3, CT47A4, CT47A5, CT47A6, CT47A7, CT47A8, CT47A9, CT47B1, CTAG1A, CTAG1B, CTAG2, CTAGE1, CTAGE5, CTAGE6P, CTAGE9, CTBP2, CTDNEP1, CTDSP2, CTDSPL2, CTLA4, CTNNA1, CTNND1, CTRB1, CTRB2, CTSL1, CTU1, CUBN, CULL CULT, CUL9, CUTA, CUX1, CXADR, CXCL1, CXCL17, CXCL2, CXCL3, CXCL5, CXCL6, CXCR1, CXCR2, CXorf40A, CXorf40B, CXorf48, CXorf49, CXorf49B, CXorf56, CXorf61, CYB5A, CYCS, CYP11B1, CYP11B2, CYP1A1, CYP1A2, CYP21A2, CYP2A13, CYP2A6, CYP2A7, CYP2B6, CYP2C18, CYP2C19, CYP2C8, CYP2C9, CYP2D6, CYP2F1, CYP3A4, CYP3A43, CYP3A5, CYP3A7, CYP3A7-CYP3AP1, CYP46A1, CYP4A11, CYP4A22, CYP4F11, CYP4F12, CYP4F2, CYP4F3, CYP4F8, CYP4Z1, CYP51A1, CYorf17, DAP3, DAPK1, DAXX, DAZ1, DAZ2, DAZ3, DAZ4, DAZAP2, DAZL, DBF4, DCAF12L1, DCAF12L2, DCAF13, DCAF4, DCAF4L1, DCAF4L2, DCAF6, DCAF8L1, DCAF8L2, DCLRE1C, DCTN6, DCUIV1D1, DCUIV1D3, DDA1, DDAH2, DDB2, DDR1, DDT, DDTL, DDX10, DDX11, DDX18, DDX19A, DDX19B, DDX23, DDX26B, DDX39B, DDX3X, DDX3Y, DDX50, DDX55, DDX56, DDX6, DDX60, DDX60L, DEF8, DEFB103A, DEFB103B, DEFB104A, DEFB104B, DEFB105A, DEFB105B, DEFB106A, DEFB106B, DEFB107A, DEFB107B, DEFB108B, DEFB130, DEFB131, DEFB4A, DEFB4B, DENND1C, DENR, DEPDC1, DERL2, DESI2, DEXI, DGCR6, DGCR6L, DGKZ, DHFR, DHERL1, DHRS2, DHRS4, DHRS4L1, DHRS4L2, DHRSX, DHX16, DHX29, DHX34, DHX40, DICER1, DIMT1, DIS3L2, DKKL1, DLEC1, DLST, DMBT1, DMRTC1, DMRTC1B, DNAH11, DNAJA1, DNAJA2, DNAJB1, DNAJB14, DNAJB3, DNAJB6, DNAJC1, DNAJC19, DNAJC24, DNAJC25-GNG10, DNAJC5, DNAJC7, DNAJC8, DNAJC9, DND1, DNM1, DOCK1, DOCK11, DOCK9, DOK1, DOM3Z, DONSON, DPCR1, DPEP2, DPEP3, DPF2, DPH3, DPM3, DPP3, DPPA2, DPPA3, DPPA4, DPPA5, DPRX, DPY19L1, DPY19L2, DPY19L3, DPY19L4, DPY30, DRAXIN, DRD5, DRG1, DSC2, DSC3, DSE, DSTN, DTD2, DTWD1, DTWD2, DTX2, DUOX1, DUOX2, DUSP12, DUSP5, DUSP8, DUT, DUXA, DYNC1I2, DYNC1LI1, DYNLT1, DYNLT3, E2F3, EBLN1, EBLN2, EBPL, ECEL1, EDDM3A, EDDM3B, EED, EEF1A1, EEF1B2, EEF1D, EEF1E1, EEF1G, EFCAB3, EFEMP1, EFTUD1, EGFL8, EGLN1, EHD1, EHD3, EHMT2, EI24, EIF1, EIFL4X, EIF2A, EIF2C1, EIF2C3, EIF2S2, EIF2S3, EIF3A, EIF3C, EIF3CL, EIF3E, EIF3F, EIF3J, EIF3L, EIF3M, EIF4A1, EIF4A2, EIF4B, EIF4E, EIF4E2, EIF4EBP1, EIF4EBP2, EIF4H, EIF5, EIF5A, EIF5A2, EIF5AL1, ELF2, ELK1, ELL2, ELM02, EMB, EMC3, EMR1, EMR2, EMR3, ENAH, ENDOD1, ENO1, ENO3, ENPEP, ENPP7, ENSA, EP300, EP400, EPB41L4B, EPB41L5, EPCAM, EPHA2, EPHB2, EPHB3, EPN2, EPN3, EPPK1, EPX, ERCC3, ERF, ERP29, ERP44, ERVV-1, ERVV-2, ESC01, ESF1, ESPL1, ESPN, ESRRA, ETF1, ETS2, ETV3, ETV3L, EVA1C, EVPL, EVPLL, EWSR1, EX005, EXOC8, EXOG, EXOSC3, EXOSC6, EXTL2, EYS, EZR, F5, F8A1, F8A2, F8A3, FABP3, FABP5, FAF2, FAHD1, FAHD2A, FAHD2B, FAM103A1, FAM104B, FA111108A1, FA111108C1, FAM111B, FAM115A, FA111115C, FA111120A, FAM120B, FAM127A, FAM127B, FAM127C, FAM131C, FAM133B, FA111136A, FAM149B1, FAM151A, FAM153A, FAM153B, FAM154B, FA111156A, FAM156B, FA111157A, FAM157B, FAM163B, FAM165B, FAM175A, FA111177A1, FA111185A, FAM186A, FA11118B1, FAM18B2, FAM190B, FAM192A, FAM197Y1, FA111197Y3, FAM197Y4, FAM197Y6, FAM197Y7, FAM197Y8, FA111197Y9, FAM203A, FAM203B, FAM204A, FAM205A, FAM206A, FAM207A, FAM209A, FAM209B, FAM20B, FAM210B, FAM213A, FAM214B, FAM218A, FAM21A, FAM21B, FAM21C, FAM220A, FAM22A, FAM22D, FAM22F, FAM22G, FAM25A, FAM25B, FAM25C, FAM25G, FAM27E4P, FAM32A, FAM35A, FAM3C, FAM45A, FAM47A, FAM47B, FAM47C, FAM47E-STBD1, FAM58A, FAM60A, FAM64A, FAM72A, FAM72B, FAM72D, FAM76A, FAM83G, FAM86A, FAM86B2, FA11186C1, FAM89B, FAM8A1, FAM90A1, FAM91A1, FAM92A1, FAM96A, FAM98B, FAM9A, FAM9B, FAM9C, FANCD2, FANK1, FAR1, FAR2, FARP1, FARSB, FASN, FASTKD1, FAT1, FAU, FBLIM1, FBP2, FBRSL1, FBXL12, FBXO25, FBXO3, FBXO36, FBXO44, FBXO6, FBXW10, FBXW11, FBXW2, FBXW4, FCF1, FCGBP, FCGR1A, FCGR2A, FCGR2B, FCGR3A, FCGR3B, FCN1, FCN2, FCRL1, FCRL2, FCRL3, FCRL4, FCRL5, FCRL6, FDPS, FDX1, FEM1A, FEN1, FER, FFAR3, FGD5, FGF7, FGFR10P2, FH, FHL1, FIGLA, FKBP1A, FKBP4, FKBP6, FKBP8, FKBP9, FKBPL, FLG, FLG2, FLI1, FLJ44635, FLNA, FLNB, FLNC, FLOT1, FLT1, FLYWCH1, FMN2, FN3K, FOLH1, FOLH1B, FOLR1, FOLR2, FOLR3, FOSL1, FOXA1, FOXA2, FOXA3, FOXD1, FOXD2, FOXD3, FOXD4L2, FOXD4L3, FOXD4L6, FOXF1, FOXF2, FOXH1, FOXN3, FOX01, FOXO3, FPR2, FPR3, FRAT2, FREM2, FRG1, FRG2, FRG2B, FRG2C, FRMD6, FRMD7, FRMD8, FRMPD2, FSCN1, FSIP2, FTH1, FTHL17, FTL, FTO, FUNDC1, FUNDC2, FUT2, FUT3, FUT5, FUT6, FXN, FXR1, FZD2, FZD5, FZD8, G2E3, G3BP1, GABARAP, GABARAPL1, GABBR1, GABPA, GABRP, GABRR1, GABRR2, GAGE1, GAGE10, GAGE12C, GAGE12D, GAGE12E, GAGE12F, GAGE12G, GAGE12H, GAGE12I, GAGE12J, GAGE13, GAGE2A, GAGE2B, GAGE2C, GAGE2D, GAGE2E, GAPDH, GAR1, GATS, GATSL1, GATSL2, GBA, GBP1, GBP2, GBP3, GBP4, GBP5, GBP6, GBP7, GCAT, GCDH, GCNT1, GCOM1, GCSH, GDI2, GEMIN7, GEMIN8, GFRA2, GGCT, GGT1, GGT2, GGT5, GGTLC1, GGTLC2, GH1, GH2, GINS2, GJA1, GJC3, GK, GK2, GLB1L2, GLB1L3, GLDC, GLOD4, GLRA1, GLRA4, GLRX, GLRX3, GLRX5, GLTP, GLTSCR2, GLUD1, GLUL, GLYATL1, GLYATL2, GLYR1, GM2A, GMCL1, GMFB, GMPS, GNA11, GNAQ, GNAT2, GNG10, GNG5, GNGT1, GNL1, GNL3, GNL3L, GNPNAT1, GOLGA2, GOLGA4, GOLGA5, GOLGA6A, GOLGA6B, GOLGA6C, GOLGA6D, GOLGA6L1, GOLGA6L10, GOLGA6L2, GOLGA6L3, GOLGA6L4, GOLGA6L6, GOLGA6L9, GOLGA7, GOLGA8H, GOLGA8J, GOLGA8K, GOLGA8O, GON4L, GOSR1, GOSR2, GOT2, GPAA1, GPANK1, GPAT2, GPATCH8, GPC5, GPCPD1, GPD2, GPHN, GPN1, GPR116, GPR125, GPR143, GPR32, GPR89A, GPR89B, GPR89C, GPS2, GPSM3, GPX1, GPX5, GPX6, GRAP, GRAPL, GRIA2, GRIA3, GRIA4, GRK6, GRM5, GRIM, GRPEL2, GSPT1, GSTA1, GSTA2, GSTA3, GSTA5, GSTM1, GSTM2, GSTM4, GSTM5, GST01, GSTT1, GSTT2, GSTT2B, GTF2A1L, GTF2H1, GTF2H2, GTF2H2C, GTF2H4, GTF2I, GTF2IRD1, GTF2IRD2, GTF2IRD2B, GTF3C6, GTPBP6, GUSB, GXYLT1, GYG1, GYG2, GYPA, GYPB, GYPE, GZMB, GZMH, H1FOO, H2AFB1, H2AFB2, H2AFB3, H2AFV, H2AFX, H2AFZ, H2BFM, H2BFWT, H3F3A, H3F3B, H3F3C, HADHA, HADHB, HARS, HARS2, HAS3, HAUS1, HA US4, HAUS6, HAVCR1, HAX1, HBA1, HBA2, HBB, HBD, HBG1, HBG2, HBS1L, HBZ, HCAR2, HCAR3, HCN2, HCN3, HCN4, HDAC1, HDGF, HDHD1, HEATR7A, HECTD4, HERC2, HIATL1, HIBCH, HIC1, HIC2, HIGD1A, HIGD2A, HINT1, HIST1H1B, HIST1H1C, HIST1H1D, HIST1H2AA, HIST1H2AB, HIST1H2AC, HIST1H2AD, HIST1H2AE, HIST1H2AG, HIST1H2AH, HIST1H2AI, HIST1H2AL, HIST1H2BB, HIST1H2BD, HIST1H2BE, HIST1H2BF, HIST1H2BH, HIST1H2BI, HIST1H2BK, HIST1H2BM, HIST1H2B1V, HIST1H2BO, HIST1H3A, HIST1H3B, HIST1H3C, HIST1H3D, HIST1H3E, HIST1H3F, HIST1H3G, HIST1H3H, HIST1H3I, HIST1H3J, HIST1H4A, HIST1H4B, HIST1H4C, HIST1H4D, HIST1H4E, HIST1H4F, HIST1H4G, HIST1H4H, HIST1H4I, HIST1H4J, HIST1H4K, HIST1H4L, HIST2H2AA3, HIST2H2AB, HIST2H2AC, HIST2H2BE, HIST2H2BF, HIST2H3A, HIST2H3D, HIST2H4A, HIST2H4B, HIST3H2BB, HIST3H3, HIST4H4, HK2, HLA-A, HLA-B, HLA-C, HLA-DMA, HLA-DMB, HLA-DOA, HLA-DOB, HLA-DPA1, HLA-DPB1, HLA-DQA1, HLA-DQA2, HLA-DQB1, HLA-DQB2, HLA-DRA, HLA-DRB1, HLA-DRB5, HLA-E, HLA-F, HLA-G, HMGA1, HMGB1, HMGB2, HMGB3, HMGCS1, HMGN1, HMGN2, HMGN3, HMGN4, HMX1, HMX3, HNRNPA1, HNRNPA3, HNRNPAB, HNRNPC, HNRNPCL1, HNRNPD, HNRNPF, HNRNPH1, HNRNPH2, HNRNPH3, HNRNPK, HNRNPL, HNRNPM, HNRNPR, HNRNPU, HNRPDL, HOMER2, HORMAD1, HOXA2, HOXA3, HOXA6, HOXA7, HOXB2, HOXB3, HOXB6, HOXB7, HOXD3, HP, HPR, HPS1, HRG, HS3ST3A1, HS3ST3B1, HS6ST1, HSD17B1, HSD17B12, HSD17B4, HSD17B6, HSD17B7, HSD17B8, HSD3B1, HSD3B2, HSF2, HSFX1, HSFX2, HSP90AA1, HSP90AB1, HSP90B1, HSPA14, HSPA1A, HSPA1B, HSPA1L, HSPA2, HSPA5, HSPA6, HSPA8, HSPA9, HSPB1, HSPD1, HSPE1, HSPE1-MOB4, HSPG2, HTN1, HTN3, HTR3C, HTR3D, HTR3E, HTR7, HYDIN, HYPK, JARS, ID2, IDH1, IDI1, IDS, IER3, IFI16, IFIHi, IFIT1, IFIT1B, IFIT2, IFIT3, IFITM3, IFNA1, IFNA10, IFNA14, IFNA16, IFNA17, IFNA2, IFNA21, IFNA4, IFNA5, IFNA6, IFNA7, IFNA8, IFT122, IFT80, IGBP1, IGF2BP2, IGF2BP3, IGFL1, IGFL2, IGFN1, IGLL1, IGLL5, IGLON5, IGSF3, IHH, IK, IKBKG, IL17RE, IL18, IL28A, IL28B, IL29, IL32, IL3RA, IL6ST, IL9R, IIVIMP1L, IMMT, IMPAL IMPACT, IMPDHL ING5, INIP, INTS4, INTS6, IPMK, IPO7, IPPK, IQCB1, IREB2, IRX2, IRX3, IRX4, IRX5, IRX6, ISCAl, ISCA2, ISG20L2, ISL1, ISL2, IST1, ISY1-RAB43, ITFG2, ITGAD, ITGAM, ITGAX, ITGB1, ITGB6, ITIH6, ITLN1, ITLN2, ITSN1, KAL1, KANK1, KANSL1, KARS, KAT7, KATNBL1, KBTBD6, KBTBD7, KCNAJ, KCNA5, KCNA6, KCNC1, KCNC2, KCNC3, KCNH2, KCNH6, KCNJ12, KCNJ4, KCNMB3, KCTD1, KCTD5, KCTD9, KDELCJ, KDM5C, KDM5D, KDM6A, KHDC1, KHDC1L, KHSRP, KIAA0020, KIAA0146, KIAA0494, KIAA0754, KIAA0895L, KIAA1143, KIAA1191, KIAA1328, KIAA1377, KIAA1462, KIAA1549L, KIAA1551, KIAA1586, KIAA1644, KIAA1671, KIAA2013, KIF1C, KIF27, KIF4A, KIF4B, KIFC1, KIR2DL1, KIR2DL3, KIR2DL4, KIR2DS4, KIR3DL1, KIR3DL2, KIR3DL3, KLF17, KLF3, KLF4, KLF7, KLF8, KLHL12, KLHL13, KLHL15, KLHL2, KLHL5, KLHL9, KLK2, KLK3, KLRC1, KLRC2, KLRC3, KLRC4, KNTC1, KPNA2, KPNA4, KPNA7, KPNB1, KRAS, KRT13, KRT14, KRT15, KRT16, KRT17, KRT18, KRT19, KRT25, KRT27, KRT28, KRT3, KRT31, KRT32, KRT33A, KRT33B, KRT34, KRT35, KRT36, KRT37, KRT38, KRT4, KRT5, KRT6A, KRT6B, KRT6C, KRT71, KRT72, KRT73, KRT74, KRT75, KRT76, KRT8, KRT80, KRT81, KRT82, KRT83, KRT85, KRT86, KRTAP1-1, KRTAP1-3, KRTAP1-5, KRTAP10-10, KRTAP10-11, KRTAP10-12, KRTAP10-2, KRTAP10-3, KRTAP10-4, KRTAP10-7, KRTAP10-9, KRTAP12-1, KRTAP12-2, KRTAP12-3, KRTAP13-1, KRTAP13-2, KRTAP13-3, KRTAP13-4, KRTAP19-1, KRTAP19-5, KRTAP2-1, KRTAP2-2, KRTAP2-3, KRTAP2-4, KRTAP21-1, KRTAP21-2, KRTAP23-1, KRTAP3-2, KRTAP3-3, KRTAP4-12, KRTAP4-4, KRTAP4-6, KRTAP4-7, KRTAP4-9, KRTAP5-1, KRTAP5-10, KRTAP5-3, KRTAP5-4, KRTAP5-6, KRTAP5-8, KRTAP5-9, KRTAP6-1, KRTAP6-2, KRTAP6-3, KRTAP9-2, KRTAP9-3, KRTAP9-6, KRTAP9-8, KRTAP9-9, LlTD1, LAGE3, LAIR1, LAIR2, LAMTOR3, LANCL3, LAP3, LAPTM4B, LARP1, LAPP1B, LARP4, LARP7, LCE1A, LCE1B, LCE1C, LCE1D, LCE1E, LCE1F, LCE2A, LCE2B, LCE2C, LCE2D, LCE3C, LCE3D, LCE3E, LCMT1, LCN1, LDHA, LDHAL6B, LDHB, LEFTY1, LEFTY2, LETM1, LGALS13, LGALS14, LGALS16, LGALS7, LGALS7B, LGALS9, LGALS9B, LGALS9C, LGMN, LGR6, LHB, LILRA1, LILRA2, LILRA3, LILRA4, LILRA5, LILRA6, LILRB1, LILRB2, LILRB3, LILRB4, LILRB5, LIMK2, LIMS1, LIN28A, LIN28B, LIN54, LLPH, LMLN, LNX1, LOC100129083, LOC100129216, LOC100129307, LOC100129636, LOC100130539, LOC100131107, LOC100131608, LOC100132154, LOC100132202, LOC100132247, LOC100132705, LOC100132858, LOC100132859, LOC100132900, LOC100133251, LOC100133267, LOC100133301, LOC100286914, LOC100287294, LOC100287368, LOC100287633, LOC100287852, LOC100288332, LOC100288646, LOC100288807, LOC100289151, LOC100289375, LOC100289561, LOC100505679, LOC100505767, LOC100505781, LOC100506248, LOC100506533, LOC100506562, LOC100507369, LOC100507607, LOC100652777, LOC100652871, LOC100652953, LOC100996256, LOC100996259, LOC100996274, LOC100996301, LOC100996312, LOC100996318, LOC100996337, LOC100996356, LOC100996369, LOC100996394, LOC100996401, LOC100996413, LOC100996433, LOC100996451, LOC100996470, LOC100996489, LOC100996541, LOC100996547, LOC100996567, LOC100996574, LOC100996594, L0C100996610, LOC100996612, LOC100996625, LOC100996631, LOC100996643, LOC100996644, LOC100996648, LOC100996675, LOC100996689, LOC100996701, LOC100996702, LOC377711, LOC388849, LOC391322, LOC391722, LOC401052, LOC402269, LOC440243, LOC440292, LOC440563, L00554223, LOC642441, LOC642643, LOC642778, LOC642799, LOC643802, LOC644634, LOC645202, LOC645359, LOC646021, LOC646670, LOC649238, LOC728026, LOC728715, LOC728728, LOC728734, LOC728741, LOC728888, LOC729020, LOC729159, LOC729162, LOC729264, LOC729458, LOC729574, LOC729587, LOC729974, LOC730058, LOC730268, LOC731932, LOC732265, LONRF2, LPA, LPCAT3, LPGAT1, LRPS, LRPSL, LRRC16B, LRRC28, LRRC37A, LRRC37A2, LRRC37A3, LRRC37B, LRRC57, LRRC59, LRRC8B, LRRFIP1, LSM12, LSM14A, LSM2, LSM3, LSP1, LTA, LTB, LUZP6, LY6G5B, LY6G5C, LY6G6C, LY6G6D, LY6G6F, LYPLA1, LYPLA2, LYRM2, LYRM5, LYST, LYZL1, LYZL2, LYZL6, MADILL MAD2L1, MAGEA10-MAGEA5, MAGEA11, MAGEA12, MAGEA2B, MAGEA4, MAGEA5, MAGEA6, MAGEA9, MAGEB2, MAGEB4, MAGEB6, MAGEC1, MAGEC3, MAGED1, MAGED2, MAGED4, MAGED4B, MAGIX, MALL, MAMDC2, MANIA1, MAN1A2, MANBAL, MANEAL, MAP1LC3B, MAP1LC3B2, MAP2K1, MAP2K2, MAP2K4, MAP3K13, MAP7, MAPK1IP1L, MAPK6, MAPK8IP1, MAPRE1, MAPT, MARC1, MARC2, MAS1L, MASP1, MAST1, MAST2, MAST3, MAT2A, MATR3, MBD3L2, MBD3L3, MBD3L4, MBD3L5, MBLAC2, MCCD1, MCF2L2, MCFD2, MCTS1, MDC1, ME1, ME2, MEAF6, MED13, MED15, MED25, MED27, MED28, MEF2A, MEF2BNB, MEIS3, MEMO1, MEP1A, MESP1, MEST, METAP2, METTL1, ME1IL15, METTL21A, METTL21D, METTL2A, METTL2B, METTL5, METTL7A, METTLE, MEX3B, MEX3D, MFAP2, MFF, MFN1, MFSD2B, MGAM, MICA, MICB, MINOS1, MIPEP, MKI67, MKI67IP, MKNK1, MKRN1, MLF1IP, MLL3, MLLT10, MLLT6, MMADHC, MMP10, MMP23B, MMP3, MOB4, MOCS1, MOCS3, MOG, MORF4L1, MORF4L2, MPEG1, MPHOSPH10, MPHOSPH8, MPO, MPP7, MPPE1, MPRIP, MPV17L, MPZL1, MR1, MRC1, MRE11A, MRFAP1, MRFAP1L1, MRGPRX2, MRGPRX3, MRGPRX4, MRPL10, MRPL11, MRPL19, MRPL3, MRPL32, MRPL35, MRPL36, MRPL45, MRPL48, MRPL50, MRPL51, MRPS10, MRPS16, MRPS17, MRPS18A, MRPS18B, MRPS18C, MRPS21, MRPS24, MRPS31, MRPS33, MRPS36, MRPS5, MRRF, MRS2, MRTO4, MS4A4A, MS4A4E, MS4A6A, MS4A6E, MSANTD2, MSANTD3, MSANTD3-TMEFF1, MSH5, MSL3, MSN, MST1, MST01, MSX2, MT1A, MT1B, MT1E, MT1F, MT1G, MT1H, MT1M, MT1X, MT2A, MTAP, MTCH1, MTFR1, MTHFD1, MTHFD1L, MTHFD2, MTIF2, MTIF3, MTMR12, MTMR9, MTRF1L, MTRNR2L1, MTRNR2L5, MTRNR2L6, MTRNR2L8, MTX1, MUC12, MUC16, MUC19, MUC20, MUC21, MUC22, MUC5B, MUC6, MX1, MX2, MXRA5, MXRA7, MYADM, MYEOV2, MYH1, MYH11, MYH13, MYH2, MYH3, MYH4, MYH6, MYH7, MYH8, MYH9, MYL12A, MYL12B, MYL6, MYL6B, MYLK, MYO5B, MZT1, MZT2A, MZT2B, NAA40, NAALAD2, NAB1, NACA, NACA2, NACAD, NACC2, NAGK, NAIP, NAMPT, NANOG, NANOGNB, NANP, NAP1L1, NAP1L4, NAPEPLD, NAPSA, NARG2, NARS, NASP, NAT1, NAT2, NAT8, NAT8B, NBAS, NBEA, NBEAL1, NBPF1, NBPF10, NBPF11, NBPF14, NBPF15, NBPF16, NBPF4, NBPF6, NBPF7, NBPF9, NBR1, NCAPD2, NCF1, NCOA4, NCOA6, NCOR1, NCR3, NDEL1, NDST3, NDST4, NDUFA4, NDUFA5, NDUFA9, NDUFAF2, NDUFAF4, NDUFB1, NDUFB3, NDUFB4, NDUFB6, NDUFB8, NDUFB9, NDUFS5, NDUFV2, NEB, NEDD8, NEDD8-MDP1, NEFH, NEFM, NEIL2, NEK2, NETO2, NEU1, NEUROD1, NEUROD2, NF1, NFE2L3, NFIC, NFIX, NFKBIL1, NFYB, NFYC, NHLH1, NHLH2, NHP2, NHP2L1, NICN1, NIF3L1, NIP7, NIPA2, NIPAL1, NIPSNAP3A, NIPSNAP3B, NKAP, NKX1-2, NLGN4X, NLGN4Y, NLRP2, NLRP5, NLRP7, NLRP9, NMD3, NME2, NMNAT1, NOB1, NOC2L, NOL11, NOLC1, NOM01, NOMO2, NOMO3, NONO, NOP10, NOP56, NOS2, NOTCH2, NOTCH2NL, NOTCH4, NOX4, NPAP1, NPEPPS, NPIP, NPIPL3, NPM1, NPSR1, NR2F1, NR2F2, NR3C1, NRBF2, NREP, NRM, NSA2, NSF, NSFL1C, NSMAF, NSRP1, NSUN5, NT5C3, NT5DC1, NTM, NTPCR, NUBP1, NUDC, NUDT10, NUDT11, NUDT15, NUDT16, NUDT19, NUDT4, NUDT5, NUFIP1, NUP210, NUP35, NUP50, NUS1, NUTF2, NXF2, NXF2B, NXF3, NXF5, NXPE1, NXPE2, NXT1, OAT, OBP2A, OBP2B, OBSCN, OCLN, OCM, OCM2, ODC1, OFD1, OGDH, OGDHL, OGFODJ, OGFR, OLA1, ONECUT1, ONECUT2, ONECUT3, OPCML, OPN1LW, OPN1MW, OPN1MW2, OR10A2, OR10A3, OR10A5, OR10A6, OR10C1, OR10G2, OR10G3, OR10G4, OR10G7, OR10G8, OR10G9, OR10H1, OR10H2, OR10H3, OR10H4, OR10H5, OR10J3, OR10J5, OR10K1, OR10K2, OR10Q1, OR11A1, OR11G2, OR11H1, OR11H12, OR11H2, OR12D2, OR12D3, OR13C2, OR13C4, OR13C5, OR13C9, OR13D1, OR1411, OR1A1, OR1A2, OR1D2, OR1D5, OR1E1, OR1E2, OR1F1, ORLI1, ORLI2, ORLI4, OR1L4, OR1L6, OR1M1, OR1S1, OR1S2, OR2A1, OR2A12, OR2A14, OR2A2, OR2A25, OR2A4, OR2A42, OR2A5, OR2A7, OR2AG1, OR2AG2, OR2B2, OR2B3, OR2B6, OR2F1, OR2F2, OR2H1, OR2H2, OR2J2, OR2J3, OR2L2, OR2L3, OR2L5, OR2L8, OR2M2, OR2M5, OR2M7, OR2S2, OR2T10, OR2T2, OR2T27, OR2T29, OR2T3, OR2T33, OR2T34, OR2T35, OR2T4, OR2T5, OR2T8, OR2V1, OR2V2, OR2W1, OR3A1, OR3A2, OR3A3, OR4A15, OR4A47, OR4C12, OR4C13, OR4C46, OR4D1, OR4D10, OR4D11, OR4D2, OR4D9, OR4F16, OR4F21, OR4F29, OR4F3, OR4K15, OR4M1, OR4M2, OR4N2, OR4N4, OR4N5, OR4P4, OR4Q3, OR51A2, OR51A4, OR52E2, OR52E6, OR52E8, OR52H1, OR5211, OR5212, OR52J3, OR52K1, OR52K2, OR52L1, OR56A1, OR56A3, OR56A4, OR56A5, OR56B4, OR5AK2, OR5B2, OR5B3, OR5D16, OR5F1, OR5H14, OR5H2, OR5H6, OR5J2, OR5L1, OR5L2, OR5M1, OR5M10, OR5M3, OR5M8, OR5P3, OR5T1, OR5T2, OR5T3, OR5V1, OR6B2, OR6B3, OR6C6, OR7A10, OR7A5, OR7C1, OR7C2, OR7G3, OR8A1, OR8B12, OR8B2, OR8B3, OR8B8, OR8G2, OR8G5, OR8H1, OR8H2, OR8H3, OR8J1, OR8J3, OR9A2, OR9A4, OR9G1, ORC3, ORM1, ORM2, OSTC, OSTCP2, OTOA, OTOP1, OTUD4, OTUD7A, OTX2, OVOS, OXCT2, OXR1, OXT, P2RX6, P2RX7, P2RY8, PA2G4, PAAF1, PABPC1, PABPC1L2A, PABPC1L2B, PABPC3, PABPC4, PABPN1, PAEP, PAFAH1B1, PAFAH1B2, PAGE1, PAGE2, PAGE2B, PAGES, PAICS, PAIP1, PAK2, PAM PANK3, PARG, PARL, PARN, PARP1, PARP4, PARP8, PATL1, PBX1, PBX2, PCBD2, PCBP1, PCBP2, PCDH11X, PCDH11Y, PCDH8, PCDHA1, PCDHA11, PCDHA12, PCDHA13, PCDHA2, PCDHA3, PCDHA5, PCDHA6, PCDHA7, PCDHA8, PCDHA9, PCDHB10, PCDHB11, PCDHB12, PCDHB13, PCDHB15, PCDHB16, PCDHB4, PCDHB8, PCDHGA1, PCDHGA11, PCDHGA12, PCDHGA2, PCDHGA3, PCDHGA4, PCDHGA5, PCDHGA7, PCDHGA8, PCDHGA9, PCDHGB1, PCDHGB2, PCDHGB3, PCDHGB5, PCDHGB7, PCGF6, PCMTD1, PCNA, PCNP, PCNT, PCSK5, PCSK7, PDAP1, PDCD2, PDCD5, PDCD6, PDCD6IP, PDCL2, PDCL3, PDE4DIP, PDIA3, PDLIM1, PDPK1, PDPR, PDSS1, PDXDC1, PDZD11, PDZK1, PEBP1, PEF1, PEPD, PERP, PEX12, PEX2, PF4, PF4V1, PFDN1, PFDN4, PFDN6, PFKFB1, PFN1, PGA3, PGA4, PGA5, PGAM1, PGAM4, PGBD3, PGBD4, PGD, PGGT1B, PGK1, PGK2, PGM5, PHAX, PHB, PHC1, PHF1, PHF10, PHF2, PHF5A, PHKA1, PHLPP2, PHOSPHO1, PI3, PI4K2A, PI4KA, PIEZO2, PIGA, PIGF, PIGH, PIGN, PIGY, PIK3CA, PIK3CD, PILRA, PINI, PIN4, PIP5K1A, PITPNB, PKD1, PKM, PKP2, PKP4, PLA2G10, PLA2G12A, PLA2G4C, PLACE, PLAC9, PLAGL2, PLD5, PLEC, PLEKHA3, PLEKHA8, PLEKHM1, PLG, PLGLB1, PLGLB2, PLIN2, PLIN4, PLK1, PLLP, PLSCR1, PLSCR2, PLXNA1, PLXNA2, PLXNA3, PLXNA4, PM20D1, PMCH, PW2, PMPCA, PMS2, PNKD, PNLIP, PNLIPRP2, PNMA6A, PNMA6B, PNMA6C, PNMA6D, PN01, PNPLA4, PNPT1, POLD2, POLE3, POLH, POLR2E, POLR2J, POLR2J2, POLR2J3, POLR2M, POLR3D, POLR3G, POLR3K, POLRMT, POM121, POM121C, POMZP3, POTEA, POTEC, POTED, POTEE, POTEF, POTEH, POTEI, POTEJ, POTEM, PO U3F1, POU3F2, PO U3F3, PO U3F4, PO U4F2, POU4F3, POU5F1, PPA1, PPAT, PPBP, PPCS, PPEF2, PPFIBP1, PPIA, PPIAL4C, PPIAL4D, PPIAL4E, PPIAL4F, PPIE, PPIG, PPIL1, PPIP5K1, PPIP5K2, PPM1A, PPP1R11, PPP1R12B, PPP1R14B, PPP1R18, PPP1R2, PPP1R26, PPP1R8, PPP2CA, PPP2CB, PPP2R2D, PPP2R3B, PPP2R5C, PPP2R5E, PPP4R2, PPP5C, PPP5D1, PPP6R2, PPP6R3, PPT2, PPY, PRADC1, PRAMEF1, PRAMEF10, PRAMEF11, PRAMEF12, PRAMEF13, PRAMEF14, PRAMEF15, PRAMEF16, PRAMEF17, PRAMEF18, PRAMEF19, PRAMEF20, PRAMEF21, PRAMEF22, PRAMEF23, PRAMEF25, PRAMEF3, PRAMEF4, PRAMEF5, PRAMEF6, PRAMEF7, PRAMEF8, PRAMEF9, PRB1, PRB2, PRB3, PRB4, PRDM7, PRDM9, PRDX1, PRDX2, PRDX3, PRDX6, PRELM1, PRG4, PRH1, PRH2, PRKAR1A, PRKCI, PRKRA, PRKRIR, PRKX, PRAIT1, PRAIT5, PRODH, PROKR1, PROKR2, PROS1, PRPF3, PRPF38A, PRPF4B, PRPS1, PRR12, PRR13, PRR20A, PRR20B, PRR20C, PRR20D, PRR20E, PRR21, PRR23A, PRR23B, PRR23C, PRR3, PRR5-ARHGAP8, PRRC2A, PRRC2C, PRRT1, PRSS1, PRSS21, PRSS3, PRSS41, PRSS42, PRSS48, PRUNE, PRY, PRY2, PSAT1, PSG1, PSG11, PSG2, PSG3, PSG4, PSG5, PSG6, PSG8, PSG9, PSIP1, PSMA6, PSMB3, PSMB5, PSMB8, PSMB9, PSMC1, PSMC2, PSMC3, PSMC5, PSMC6, PSMD10, PSMD12, PSMD2, PSMD4, PSMD7, PSMD8, PSME2, PSORS1C1, PSORS1C2, PSPH, PTBP1, PTCD2, PTCH1, PTCHD3, PTCHD4, PTEN, PTGES3, PTGES3L-AARSD1, PTGR1, PTMA, PTMS, PTOV1, PTP4A1, PTP4A2, PTPN11, PTPN2, PTPN20A, PTPN20B, PTPRD, PTPRH, PTPRM PTPRN2, PTPRU, PTTG1, PTTG2, PVRIG, PVRL2, PWWP2A, PYGB, PYGL, PYHIN1, PYROXD1, PYURF, PYY, PZP, QRSL1, R3HDM2, RAB11A, RAB11FIP1, RAB13, RAB18, RAB1A, RAB1B, RAB28, RAB31, RAB40AL, RAB40B, RAB42, RAB43, RAB5A, RAB5C, RAB6A, RAB6C, RAB9A, RABGEFJ, RABGGTB, RABL2A, RABL2B, RABL6, RAC1, RACGAP1, RAD1, RAD17, RAD21, RAD23B, RAD51AP1, RAD54L2, RAET1G, RAET1L, RALA, RALBP1, RALGAPA1, RAN, RANBP1, RANBP17, RANBP2, RANBP6, RAP1A, RAP1B, RAP1GDS1, RAP2A, RAP2B, RARS, RASA4, RASA4B, RASGRP2, RBAK, RBAK-LOC389458, RBBP4, RBBP6, RBM14-RBM4, RBM15, RBM17, RBM39, RBM4, RBM43, RBM48, RBM4B, RBM7, RBM8A, RBMS1, RBMS2, RBMX, RBMX2, RBMXLJ, RBMXL2, RBMY1A1, RBMY1B, RBMY1D, RBMY1E, RBMY1F, RBMYLI, RBPJ, RCBTB1, RCBTB2, RCC2, RCN1, RCOR2, RDBP, RDH16, RDM1, RDX, RECQL, REGIA, REG1B, REG3A, REG3G, RELA, RERE, RETSAT, REV1, REXO4, RFC3, RFESD, RFK, RFPL1, RFPL2, RFPL3, RFPL4A, RFTN1, RFWD2, RGL2, RGPD1, RGPD2, RGPD3, RGPD4, RGPD5, RGPD6, RGPD8, RGS17, RGS19, RGS9, RHBDFJ, RHCE, RHD, RHEB, RHOQ, RHOT1, RHOXF2, RHOXF2B, RHPN2, RIMBP3, RIMBP3B, RIMBP3C, RIMKLB, RING1, RLIA1, RLN1, RLN2, RLTPR, RMND1, RMND5A, RNASE2, RNASE3, RNASE7, RNASE8, RNASEHJ, RNASET2, RNF11, RNF123, RNF126, RNF13, RNF138, RNF14, RNF141, RNF145, RNF152, RNF181, RNF2, RNF216, RNF39, RNF4, RNF5, RNF6, RNFT1, RNMTL1, RNPC3, RNPS1, ROBO2, ROCK1, ROCK2, ROPN1, ROPN1B, RORA, RP9, RPA2, RPA3, RPAP2, RPE, RPF2, RPGR, RPL10, RPL10A, RPL10L, RPL12, RPL13, RPL14, RPL15, RPL17, RPL17-C180RF32, RPL18A, RPL19, RPL21, RPL22, RPL23, RPL23A, RPL24, RPL26, RPL26L1, RPL27, RPL27A, RPL29, RPL3, RPL30, RPL31, RPL32, RPL35, RPL35A, RPL36, RPL36A, RPL36A-HNRNPH2, RPL36AL, RPL37, RPL37A, RPL39, RPL4, RPL41, RPL5, RPL6, RPL7, RPL7A, RPL7L1, RPL8, RPL9, RPLP0, RPLP1, RPP21, RPS10, RPS10-NUDT3, RPS11, RPS13, RPS14, RPS15, RPS15A, RPS16, RPS17, RPS17L, RPS18, RPS19, RPS2, RPS20, RPS23, RPS24, RPS25, RPS26, RPS27, RPS27A, RPS28, RPS3, RPS3A, RPS4X, RPS4Y1, RPS4Y2, RPS5, RPS6, RPS6KB1, RPS7, RPS8, RPS9, RPSA, RPTN, RRAGA, RRAGB, RRAS2, RRA12, RRN3, RRP7A, RSL24D1, RSPH10B, RSPH10B2, RSPO2, RSRC1, RSUl, RTEL1, RTN3, RTN4IP1, RTN4R, RTP1, RTP2, RUFY3, RUNDC1, RUVBL2, RWDD1, RWDD4, RXRB, RYK, S100A11, S100A7L2, SAA1, SAA2, SAA2-SAA4, SAE1, SAFB, SAFB2, SAGE1, SALL1, SALL4, SAMD12, SAMD9, SAMD9L, SAP18, SAP25, SAP30, SAPCD1, SAPCD2, SAR1A, SATL1, SAV1, SAYSD1, SBDS, SBF1, SCAMPI, SCAND3, SCD, SCGB1D1, SCGB1D2, SCGB1D4, SCGB2A1, SCGB2A2, SCGB2B2, SCN10A, SCN1A, SCN2A, SCN3A, SCN4A, SCN5A, SCN9A, SCOC, SCXA, SCXB, SCYL2, SDAD1, SDCBP, SDCCAG3, SDHA, SDHB, SDHC, SDHD, SDR42E1, SEC11A, SEC14L1, SEC14L4, SEC14L6, SEC61B, SEC63, SELT, SEMA3E, SEA1G1, SEMG2, SEPHS1, SEPHS2, SEPT14, SEPTI, SERBP1, SERF1A, SERF1B, SERF2, SERHL2, SERPINB3, SERPINB4, SERPINH1, SET, SETD8, SF3A2, SF3A3, SF3B14, SF3B4, SFR1, SFRP4, SFTA2, SFTPA1, SFTPA2, SH2D1B, SH3BGRL3, SH3GL1, SHANK2, SHC1, SHCBP1, SHFM1, SHH, SHISA5, SHMT1, SHOX, SHQ1, SHROOM2, SIGLEC10, SIGLEC11, SIGLEC12, SIGLEC14, SIGLEC5, SIGLEC6, SIGLEC7, SIGLEC8, SIGLEC9, SIMC1, SIN3A, SIRPA, SIRPB1, SIRPG, SIX1, SIX2, SKA2, SKIV2L, SKOR2, SKP1, SKP2, SLAIN2, SLAMF6, SLC10A5, SLC16A14, SLC16A6, SLC19A3, SLC22A10, SLC22A11, SLC22A12, SLC22A24, SLC22A25, SLC22A3, SLC22A4, SLC22A5, SLC22A9, SLC25A13, SLC25A14, SLC25A15, SLC25A20, SLC25A29, SLC25A3, SLC25A33, SLC25A38, SLC25A47, SLC25A5, SLC25A52, SLC25A53, SLC25A6, SLC29A4, SLC2A13, SLC2A14, SLC2A3, SLC31A1, SLC33A1, SLC35A4, SLC35E1, SLC35E2, SLC35E2B, SLC35G3, SLC35G4, SLC35G5, SLC35G6, SLC36A1, SLC36A2, SLC39A1, SLC39A7, SLC44A4, SLC4A1AP, SLC52A1, SLC52A2, SLC5A6, SLC5A8, SLC6A14, SLC6A6, SLC6A8, SLC7A5, SLC8A2, SLC8A3, SLC9A2, SLC9A4, SLC9A7, SLCO1B1, SLCO1B3, SLCO1B7, SLFN11, SLFN12, SLFN12L, SLFN13, SLFN5, SLIRP, SLM02, SLX1A, SLX1B, SMARCE1, SMC3, SMC5, SMEK2, SMG1, SMIV1, SMN2, SMR3A, SMR3B, SMS, SMUT, SMURF2, SNAIL SNAPC4, SNAPC5, SNF8, SNRNP200, SNRPA1, SNRPB2, SNRPC, SNRPD1, SNRPD2, SNRPE, SNRPG, SNRPN, SNW1, SNX19, SNX25, SNX29, SNX5, SNX6, SOCS5, SOCS6, SOGA1, SOGA2, SON, SOX1, SOX10, SOX14, SOX2, SOX30, SOX5, SOX9, SP100, SP140, SP140L, SP3, SP5, SP8, SP9, SPACA5, SPACA5B, SPACA7, SPAG11A, SPAG11B, SPANXA1, SPANXB1, SPANXD, SPANXN2, SPANXN5, SPATA16, SPATA20, SPATA31A1, SPATA31A2, SPATA31A3, SPATA31A4, SPATA31A5, SPATA31A6, SPATA31A7, SPATA31C1, SPATA31C2, SPATA31D1, SPATA31D3, SPATA31D4, SPATA31E1, SPCS2, SPDYE1, SPDYE2, SPDYE2L, SPDYE3, SPDYE4, SPDYE5, SPDYE6, SPECC1, SPECC1L, SPHAR, SPIC, SPIN1, SPIN2A, SPIN2B, SPOPL, SPPL2A, SPPL2C, SPR, SPRR1A, SPRR1B, SPRR2A, SPRR2B, SPRR2D, SPRR2E, SPRR2F, SPRY3, SPRYD4, SPTLC1, SRD5A1, SRD5A3, SREK1IP1, SRGAP2, SRP14, SRP19, SRP68, SRP72, SRP9, SRPK1, SRPK2, SRRI111, SRSF1, SRSF10, SRSF11, SRSF3, SRSF6, SRSF9, SRXN1, SS18L2, SSB, SSBP2, SSBP3, SSBP4, SSNA1, SSR3, SSX1, SSX2, SSX2B, SSX3, SSX4, SSX4B, SSX5, SSX7, ST13, ST3GAL1, STAG3, STAR, STAT5A, STAT5B, STAU1, STAU2, STBD1, STEAP1, STEAP1B, STH, STIP1, STK19, STK24, STK32A, STMIV1, STMN2, STMN3, STRADB, STRAP, STRC, STRN, STS, STUB1, STX18, SUB1, SUCLA2, SUCLG2, SUDS3, SUGP1, SUGT1, SULT1A1, SULT1A2, SULT1A3, SULT1A4, SUMF2, SUMO1, SUMO2, SUPT16H, SUPT4H1, SUSD2, SUZ12, SVIL, SWI5, SYCE2, SYNCRIP, SYNGAP1, SYNGR2, SYT14, SYT15, SYT2, SYT3, SZRD1, TAAR6, TAAR8, TACC1, TADA1, TAF1, TAF15, TAF1L, TAF4B, TAF5L, TAF9, TAF9B, TAGLN2, TALD01, TANC2, TAP1, TAP2, TAPBP, TARBP2, TARDBP, TARP, TAS2R19, TAS2R20, TAS2R30, TAS2R39, TAS2R40, TAS2R43, TAS2R46, TAS2R50, TASP1, TATDN1, TATDN2, TBC1D26, TBC1D27, TBC1D28, TBC1D29, TBC1D2B, TBC1D3, TBC1D3B, TBC1D3C, TBC1D3F, TBC1D3G, TBC1D3H, TBCA, TBCCD1, TBL1X, TBL1XR1, TBL1Y, TBPL1, TBX20, TC2N, TCEA1, TCEAL2, TCEAL3, TCEAL5, TCEB1, TCEB2, TCEB3B, TCEB3C, TCEB3CL, TCEB3CL2, TCERG1L, TCF19, TCF3, TCHH, TCL1B, TCOF1, TCP1, TCP10, TCP10L, TCP10L2, TDG, TDGF1, TDRD1, TEAD1, TEC, TECR, TEKT4, TERF1, TERF2IP, TET1, TEX13A, TEX13B, TEX28, TF, TFB2M, TFDP3, TFG, TGIF1, TGIF2, TGIF2LX, TGIF2LY, THAP3, THAP5, THEM4, THOC3, THRAP3, THSD1, THUMPD1, TIMM17B, TIMM23B, TIMM8A, TIMM8B, TIMP4, TIPIN, TJAP1, TJP3, TLE1, TLE4, TLK1, TLK2, TLL1, TLR1, TLR6, TMA16, TMA7, TMC6, TMCC1, TMED10, TMED2, TMEM126A, TMEM128, TMEM132B, TMEM132C, TMEM14B, TMEM14C, TMEM161B, TMEM167A, TMEM183A, TMEM183B, TMEM185A, TMEM185B, TMEM189-UBE2V1, TMEM191B, TMEM191C, TMEM230, TMEM231, TMEM236, TMEM242, TMEM251, TMEM254, TMEM30B, TMEM47, TMEM69, TMEM80, TMEM92, TMEM97, TMEM98, TMLHE, TMPRSS11E, TMSB10, TMSB15A, TMSB15B, TMSB4X, TMSB4Y, TMTC1, TMTC4, TMX1, TMX2, TNC, TNF, TNFRSF10A, TNFRSF10B, TNFRSF10C, TNFRSF10D, TNFRSF13B, TNFRSF14, TNIP2, TNN, TNP01, TNRC18, TNXB, TOB2, TOE1, TOW20, TOW40, TOMM6, TOW7, TOP1, TOP3B, TOR1B, TOR3A, TOX4, TP53TG3, TP53TG3B, TP53TG3C, TPD52L2, TPI1, TPM3, TPM4, TPMT, TPRKB, TPRX1, TPSAB1, TPSB2, TPSD1, TPT1, TPTE, TPTE2, TRA2A, TRAF6, TRAPPC2, TRAPPC2L, TREH, TREML2, TREML4, TRIM10, TRIM15, TRIM16, TRIM16L, TRIM26, TRIM27, TRIM31, TRIM38, TRIM39, TRIM39-RPP21, TRIM40, TRIM43, TRIM43B, TRIM48, TRIM49, TRIM49B, TRIM49C, TRIM49DP, TRIM49L1, TRIM50, TRIM51, TRIM51GP, TRIM60, TRIM61, TRIM64, TRIM64B, TRIM64C, TRIM73, TRIM74, TRIM77P, TRIP11, TRMT1, TRMT11, TRMT112, TRMT2B, TRNT1, TRO, TRPA1, TRPC6, TRPV5, TRPV6, TSC22D3, TSEN15, TSEN2, TSPA1V11, TSPY1, TSPY10, TSPY2, TSPY3, TSPY4, TSPY8, TSPYL1, TSPYL6, TSR1, TSSK1B, TSSK2, TTC28, TTC3, TTC30A, TTC30B, TTC4, 11L, TTLL12, TTLL2, TTN, TUBA1A, TUBA1B, TUBA1C, TUBA3C, TUBA3D, TUBA3E, TUBA4A, TUBAE, TUBB, TUBB2A, TUBB2B, TUBB3, TUBB4A, TUBB4B, TUBB6, TUBB8, TUBE1, TUBG1, TUBG2, TUBGCP3, TUBGCP6, TUFM, TWF1, TWIST2, TXLNG, TXN2, TXNDC2, TXNDC9, TYR, TYRO3, TYW1, TYW1B, U2AF1, UAP1, UBA2, UBA5, UBD, UBE2C, UBE2D2, UBE2D3, UBE2D4, UBE2E3, UBE2F, UBE2H, UBE2L3, UBE2M, UBE2N, UBE2Q2, UBE2S, UBE2V1, UBE2V2, UBE2W, UBE3A, UBFD1, UBQLNJ, UBQLN4, UBTFL1, UBXN2B, UFD1L, UFM1, UGT1A10, UGT1A3, UGT1A4, UGT1A5, UGT1A7, UGT1A8, UGT1A9, UGT2A1, UGT2A2, UGT2A3, UGT2B10, UGT2B11, UGT2B15, UGT2B17, UGT2B28, UGT2B4, UGT2B7, UGT3A2, UHRF1, UHRF2, ULBP1, ULBP2, ULBP3, ULK4, UNC93A, UNC93B1, UPF3A, UPK3B, UPK3BL, UQCR10, UQCRB, UQCRFS1, UQCRH, UQCRQ, USP10, USP12, USP13, USP17L10, USP17L11, USP17L12, USP17L13, USP17L15, USP17L17, USP17L18, USP17L19, USP17L1P, USP17L2, USP17L20, USP17L21, USP17L22, USP17L24, USP17L25, USP17L26, USP17L27, USP17L28, USP17L29, USP17L3, USP17L30, USP17L4, USP17L5, USP17L7, USP17L8, USP18, USP22, USP32, USP34, USP6, USP8, USP9X, USP9Y, UTP14A, UTP14C, UTP18, UTP6, VAMPS, VAMP7, VAPA, VARS, VARS2, VCX, VCX2, VCX3A, VCX3B, VCY, VCY1B, VDAC1, VDAC2, VDAC3, VENTX, VEZF1, VKORC1, VKORC1L1, VMA21, V1V1R4, VNN1, VOPP1, VPS26A, VPS35, VPS37A, VPS51, VPS52, VSIG10, VTCN1, VTI1B, VWA5B2, VWA7, VWA8, VWF, WARS, WASF2, WASF3, WASH1, WBP1, WBP11, WBP1L, WBSCR16, WDR12, WDR45, WDR45L, WDR46, WDR49, WDR59, WDR70, WDR82, WDR89, WFDC10A, WFDC10B, WHAIVIM, WHSC1L1, WIPI2, WIZ, WNT3, WNT3A, WNT5A, WNT5B, WNT9B, WRN, WTAP, WWC2, WWC3, WWP1, XAGE1A, XAGE1B, XAGE1C, XAGE1D, XAGE1E, XAGE2, XAGE3, XAGE5, XBP1, XCL1, XCL2, XG, XIAP, XKR3, XKR8, XKRY, XKRY2, XPO6, XPOT, XRCC6, YAP1, YBX1, YBX2, YES1, YME1L1, YPEL5, YTHDC1, YTHDF1, YTHDF2, YWHAB, YWHAE, YWHAQ, YWHAZ, YY1, YY1AP1, ZAN, ZBED1, ZBTB10, ZBTB12, ZBTB22, ZBTB44, ZBTB45, ZBTB8OS, ZBTB9, ZC3H11A, ZC3H12A, ZCCHC10, ZCCHC12, ZCCHC17, ZCCHC18, ZCCHC2, ZCCHC7, ZCCHC9, ZCRB1, ZDHHC11, ZDHHC20, ZDHHC3, ZDHHC8, ZEB2, ZFAND5, ZFAND6, ZFP106, ZFP112, ZFP14, ZFP57, ZFP64, ZFP82, ZFR, ZFX, ZFY, ZFYVE1, ZFYVE9, ZIC1, ZIC2, ZIC3, ZIC4, ZIK1, ZKSCAN3, ZKSCAN4, ZMIZ1, ZAKZ2, ZMYM2, ZMYM5, ZNF100, ZNF101, ZNF107, ZNF114, ZNF117, ZNF12, ZNF124, ZNF131, ZNF135, ZNF14, ZNF140, ZNF141, ZNF146, ZNF155, ZNF160, ZNF167, ZNF17, ZNF181, ZNF185, ZNF20, ZNF207, ZNF208, ZNF212, ZNF221, ZNF222, ZNF223, ZNF224, ZNF225, ZNF226, ZNF229, ZNF230, ZNF233, ZNF234, ZNF235, ZNF248, ZNF253, ZNF254, ZNF257, ZNF259, ZNF26, ZNF264, ZNF266, ZNF267, ZNF280A, ZNF280B, ZNF282, ZNF283, ZNF284, ZNF285, ZNF286A, ZNF286B, ZNF300, ZNF302, ZNF311, ZNF317, ZNF320, ZNF322, ZNF323, ZNF324, ZNF324B, ZNF33A, ZNF33B, ZNF341, ZNF347, ZNF35, ZNF350, ZNF354A, ZNF354B, ZNF354C, ZNF366, ZNF37A, ZNF383, ZNF396, ZNF41, ZNF415, ZNF416, ZNF417, ZNF418, ZNF419, ZNF426, ZNF429, ZNF43, ZNF430, ZNF431, ZNF433, ZNF439, ZNF44, ZNF440, ZNF441, ZNF442, ZNF443, ZNF444, ZNF451, ZNF460, ZNF468, ZNF470, ZNF479, ZNF480, ZNF484, ZNF486, ZNF491, ZNF492, ZNF506, ZNF528, ZNF532, ZNF534, ZNF543, ZNF546, ZNF547, ZNF548, ZNF552, ZNF555, ZNF557, ZNF558, ZNF561, ZNF562, ZNF563, ZNF564, ZNF57, ZNF570, ZNF578, ZNF583, ZNF585A, ZNF585B, ZNF586, ZNF587, ZNF587B, ZNF589, ZNF592, ZNF594, ZNF595, ZNF598, ZNF605, ZNF607, ZNF610, ZNF613, ZNF614, ZNF615, ZNF616, ZNF620, ZNF621, ZNF622, ZNF625, ZNF626, ZNF627, ZNF628, ZNF646, ZNF649, ZNF652, ZNF655, ZNF658, ZNF665, ZNF673, ZNF674, ZNF675, ZNF676, ZNF678, ZNF679, ZNF680, ZNF681, ZNF682, ZNF69, ZNF700, ZNF701, ZNF705A, ZNF705B, ZNF705D, ZNF705E, ZNF705G, ZNF706, ZNF708, ZNF709, ZNF710, ZNF714, ZNF716, ZNF717, ZNF718, ZNF720, ZNF721, ZNF726, ZNF727, ZNF728, ZNF729, ZNF732, ZNF735, ZNF736, ZNF737, ZNF746, ZNF747, ZNF749, ZNF75A, ZNF75D, ZNF761, ZNF763, ZNF764, ZNF765, ZNF766, ZNF770, ZNF773, ZNF775, ZNF776, ZNF777, ZNF780A, ZNF780B, ZNF782, ZNF783, ZNF791, ZNF792, ZNF799, ZNF805, ZNF806, ZNF808, ZNF812, ZNF813, ZNF814, ZNF816, ZNF816-ZNF321P, ZNF823, ZNF829, ZNF83, ZNF836, ZNF84, ZNF841, ZNF844, ZNF845, ZNF850, ZNF852, ZNF878, ZNF879, ZNF880, ZNF90, ZNF91, ZNF92, ZNF93, ZNF98, ZNF99, ZNRD1, ZNRF2, ZP3, ZRSR2, ZSCAN5A, ZSCAN5B, ZSCAN5D, ZSWIM5, ZXDA, ZXDB, ZXDC, portions thereof, modified forms thereof or combinations thereof. In certain embodiments, a desired nucleic acid or gene is selected from one or more of ANGPTL4, APOB, APOC3, ASGR1, CD19, CD36, G6PC, PCSK9, EYA4, GJB2, SLC26A4, ABCA4, CNGA3, CNGB3, MERTK, MYO7A, REP1, RHO, RPE65, RS1, USH2A, PD1, PDL1, EGFR, RAF, RAS, portions thereof, and modified forms thereof.

In certain embodiments a method of editing a genome of an organism or cell comprises contacting one or more cells with one or more nucleic acids, or a composition described herein. In certain embodiments a method of editing a genome of an organism or cell is a method of modifying a target sequence in a genome of a cell, organism or subject. In certain embodiments a method of editing a genome of an organism or cell comprises introducing one or more nucleic acids described herein into one or more cells. One or more nucleic acid can be introduced into one or more cells by any suitable method.

In some embodiments, a method described herein comprises contacting a cell, or introducing into a cell, (i) a nucleic acid configured to express an Argonaute polypeptide or a functional fragment of an Argonaute polypeptide and (ii) one or more oligonucleotide guides. In some embodiments one guide oligonucleotide is contacted with or introduced into a cell or organism. In certain embodiments, two or more different oligonucleotide guides are contacted with or introduced into a cell or organism. In some embodiments, the method further comprises contacting the cell, or introducing into the cell, a donor nucleic acid (a donor sequence). In some embodiments, a donor nucleic acid comprises a desired nucleic acid flanked by a 5′-flanking sequence and a 3′ flanking sequence. In some embodiments, a donor nucleic acid may comprise a desired nucleic acid flanked by a sequence of the one or more guide oligonucleotides. In some embodiments, a method described herein comprises contacting a cell, or introducing into a cell, (i) a nucleic acid encoding an Argonaute polypeptide or a functional fragment of an Argonaute polypeptide (e.g., a nucleic acid of SEQ ID NO:1), (ii) one or more oligonucleotide guides, and (iii) a donor nucleic acid. In certain embodiments the donor nucleic acid sequence comprises a desired nucleic acid. In some embodiments, the method induces, results in, or provides a modification of a target sequence. A modification of a target sequence may comprise an insertion, deletion or replacement of one or more nucleotides of the target sequence. In some embodiments, a modification of a target sequence comprises an insertion, deletion or replacement of a single nucleotide of the target sequence. In certain embodiments, the method results in integration or insertion of a desired nucleic acid into the genome of the cell. In certain embodiments, the method results in replacement of a dysfunctional or mutated endogenous gene, or portion thereof, in the genome of a cell, with a wild-type, modified and/or a more functional gene. In certain embodiments, the method results in targeted disruption of an endogenous or wild type gene in the genome of the cell.

A cell may be contacted with a nucleic acid encoding an Argonaute, a guide oligonucleotide and/or a donor sequence at the same time, or at different times. For example, a cell may be contacted with a nucleic acid encoding an Argonaute, followed by contacting the cell with a guide oligonucleotide and/or a donor sequence within a time range of a week, 0 to 72 hours, 0 to 24 hours, 0 to 12 hours, 0 to 6 hours or 0 to 4 hours. A cell may be contacted with the nucleic acids described herein in any order.

### Pharmaceutical Compositions

In some embodiments a pharmaceutical composition comprises one or more nucleic acids described herein.

In certain embodiments, acceptable pharmaceutical compositions are nontoxic to recipients at the dosages and concentrations employed. A pharmaceutical composition can be formulated for a suitable route of administration. In some embodiments a pharmaceutical composition is formulated for subcutaneous (s.c.), intradermal, intramuscular, intraperitoneal and/or intravenous (i.v.) administration. In certain embodiments, a pharmaceutical composition can contain formulation materials for modifying, maintaining, or preserving, for example, the pH, osmolarity, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates (e.g., phosphate buffered saline) or suitable organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counter ions (such as sodium); solvents (such as glycerin, propylene glycol or polyethylene glycol); diluents; excipients and/or pharmaceutical adjuvants (Remington's Pharmaceutical Sciences, 18th Ed., A. R. Gennaro, ed., Mack Publishing Company (1995)).

In certain embodiments, a pharmaceutical composition comprises a suitable excipient, non-limiting example of which include anti-adherents (e.g., magnesium stearate), binders, fillers, monosaccharides, disaccharides, other carbohydrates (e.g., glucose, mannose or dextrins), sugar alcohols (e.g., mannitol or sorbitol), coatings (e.g., cellulose, hydroxypropyl methylcellulose (HPMC), microcrystalline cellulose, synthetic polymers, shellac, gelatin, corn protein zein, enterics or other polysaccharides), starch (e.g., potato, maize or wheat starch), silica, colors, disintegrants, flavors, lubricants, preservatives, sorbents, sweetners, vehicles, suspending agents, surfactants and/or wetting agents (such as pluronics, PEG, sorbitan esters, polysorbates such as polysorbate 20, polysorbate 80, triton, tromethamine, lecithin, cholesterol, tyloxapal), stability enhancing agents (such as sucrose or sorbitol), and tonicity enhancing agents (such as alkali metal halides, sodium or potassium chloride, mannitol, sorbitol), and/or any excipient disclosed in Remington's Pharmaceutical Sciences, 18th Ed., A. R. Gennaro, ed., Mack Publishing Company (1995).

In some embodiments a pharmaceutical composition comprises a suitable pharmaceutically acceptable additive and/or carrier. Non-limiting examples of suitable additives include a suitable pH adjuster, a soothing agent, a buffer, a sulfur-containing reducing agent, an antioxidant and the like. Non-limiting examples of sulfur-containing reducing agents include those having a sulfhydryl group such as N-acetylcysteine, N-acetylhomocysteine, thioctic acid, thiodiglycol, thioethanolamine, thioglycerol, thiosorbitol, thioglycolic acid and a salt thereof, sodium thiosulfate, glutathione, and a C1-C7 thioalkanoic acid. Non-limiting examples of an antioxidant include erythorbic acid, dibutylhydroxytoluene, butylhydroxyanisole, alpha-tocopherol, tocopherol acetate, L-ascorbic acid and a salt thereof, L-ascorbyl palmitate, L-ascorbyl stearate, sodium bisulfite, sodium sulfite, triamyl gallate and propyl gallate, as well as chelating agents such as disodium ethylenediaminetetraacetate (EDTA), sodium pyrophosphate and sodium metaphosphate. Furthermore, diluents, additives and excipients may comprise other commonly used ingredients, for example, inorganic salts such as sodium chloride, potassium chloride, calcium chloride, sodium phosphate, potassium phosphate and sodium bicarbonate, as well as organic salts such as sodium citrate, potassium citrate and sodium acetate.

The pharmaceutical compositions used herein can be stable over an extended period of time, for example on the order of months or years. In some embodiments a pharmaceutical composition comprises one or more suitable preservatives. Non limiting examples of preservatives include benzalkonium chloride, benzoic acid, salicylic acid, thimerosal, phenethyl alcohol, methylparaben, propylparaben, chlorhexidine, sorbic acid, hydrogen peroxide, the like and/or combinations thereof. A preservative can comprise a quaternary ammonium compound, such as benzalkonium chloride, benzoxonium chloride, benzethonium chloride, cetrimide, sepazonium chloride, cetylpyridinium chloride, or domiphen bromide (BRADOSOL®). A preservative can comprise an alkyl-mercury salt of thiosalicylic acid, such as thimerosal, phenylmercuric nitrate, phenylmercuric acetate or phenylmercuric borate. A preservative can comprise a paraben, such as methylparaben or propylparaben. A preservative can comprise an alcohol, such as chlorobutanol, benzyl alcohol or phenyl ethyl alcohol. A preservative can comprise a biguanide derivative, such as chlorohexidine or polyhexamethylene biguanide. A preservative can comprise sodium perborate, imidazolidinyl urea, and/or sorbic acid. A preservative can comprise stabilized oxychloro complexes, such as known and commercially available under the trade name PURITE®. A preservative can comprise polyglycol-polyamine condensation resins, such as known and commercially available under the trade name POLYQUART® from Henkel KGaA. A preservative can comprise stabilized hydrogen peroxide. A preservative can be benzalkonium chloride. In some embodiments a pharmaceutical composition is free of preservatives.

In some embodiments a pharmaceutical composition is substantially free of serum proteins. In some embodiments a pharmaceutical composition is sterile. In some embodiments a pharmaceutical composition is lyophilized to a dry powder form, which is suitable for reconstitution with a suitable pharmaceutical solvent (e.g., water, saline, an isotonic buffer solution (e.g., PBS), and the like), which reconstituted form is suitable for parental administration (e.g., intravenous administration) to a mammal.

The pharmaceutical compositions described herein may be configured for administration to a subject in any suitable form and/or amount according to the therapy in which they are employed. For example, a pharmaceutical composition configured for parenteral administration (e.g., by injection or infusion), may take the form of a suspension, solution or emulsion in an oily or aqueous vehicle and it may contain formulation agents, excipients, additives and/or diluents such as aqueous or non-aqueous solvents, co-solvents, suspending solutions, preservatives, stabilizing agents and or dispersing agents.

In some embodiments a pharmaceutical compositions described herein may be configured for administration by any suitable route of administration and may include one or more of a binding and/or lubricating agent, polymeric glycols, gelatins, cocoa-butter or other suitable waxes or fats. In some embodiments, a pharmaceutical composition described herein is incorporated into a topical formulation containing a topical carrier that is generally suited to topical drug administration and comprising any suitable material known in the art. A topical carrier may be selected so as to provide the composition in the desired form, e.g., as a solution or suspension, an ointment, a lotion, a cream, a salve, an emulsion or microemulsion, a gel, an oil, a powder, or the like. It may be comprised of naturally occurring or synthetic materials, or both. A carrier for the active ingredient may also be in a spray form. It is preferable that the selected carrier not adversely affect the active agent or other components of the topical formulation. Non-limiting examples of suitable topical carriers for use herein can be soluble, semi-solid or solid and include water, alcohols and other nontoxic organic solvents, glycerin, mineral oil, silicone, petroleum jelly, lanolin, fatty acids, vegetable oils, parabens, waxes, and the like. Semisolid carriers preferably have a dynamic viscosity greater than that of water. Other suitable vehicles include ointment bases, conventional creams such as HEB cream; gels; as well as petroleum jelly and the like. If desired, and depending on the carrier, the compositions may be sterilized or mixed with auxiliary agents, e.g., preservatives, stabilizers, wetting agents, buffers, or salts for influencing osmotic pressure and the like. Formulations may be colorless, odorless ointments, lotions, creams, microemulsions and gels.

Ointments can be semisolid preparations which are typically based on petrolatum or other petroleum derivatives. The specific ointment base to be used, as will be appreciated by those skilled in the art, is one that will provide for optimum delivery of the active agent, and, preferably, will provide for other desired characteristics as well, e.g., emolliency or the like. As with other carriers or vehicles, an ointment base should be inert, stable, nonirritating and nonsensitizing. Ointment bases can be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (OAV) emulsions, and include, for example, cetyl alcohol, glyceryl monostearate, lanolin and stearic acid. Exemplary water-soluble ointment bases are prepared from polyethylene glycols (PEGs) of varying molecular weight, e.g., polyethylene glycol-1000 (PEG-1000). Oils of animal or plant origin such as peanut oil, mineral oil, soybean oil, or sesame oil, corn oil, or synthetic oils may be added.

Nucleic acids and/or peptides may be incorporated into lotions, which generally are preparations to be applied to the skin surface without friction, and are typically liquid or semiliquid preparations in which solid particles, including the active agent, are present in a water or alcohol base. Lotions can be suspensions of solids, and may comprise a liquid oily emulsion of the oil-in-water type. In certain embodiments, lotions are preferred formulations for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethylcellulose, or the like. In some embodiments a lotion formulation for use in conjunction with the present method contains propylene glycol mixed with a hydrophilic petrolatum.

In some embodiments pharmaceutical compositions are formulated as creams, which generally are viscous liquid or semisolid emulsions, either oil-in-water or water-in-oil. Cream bases are water-washable, and contain an oil phase, an emulsifier and an aqueous phase. The oil phase is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation can be a nonionic, anionic, cationic or amphoteric surfactant.

Pharmaceutical compositions can be formulated as microemulsions, which generally are thermodynamically stable, isotropic clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, surfactant (emulsifier), co-surfactant (co-emulsifier), an oil phase and a water phase are necessary. Suitable surfactants include any surfactants that are useful in the preparation of emulsions, e.g., emulsifiers that are typically used in the preparation of creams. The co-surfactant (or “co-emulsifier”) is generally selected from the group of polyglycerol derivatives, glycerol derivatives and fatty alcohols. In some embodiments emulsifier/co-emulsifier combinations are selected from the group consisting of: glyceryl monostearate and polyoxyethylene stearate; polyethylene glycol and ethylene glycol palmitostearate; and caprylic and capric triglycerides and oleoyl macrogolglycerides. In certain embodiments a water phase includes not only water, but also, typically, buffers, glucose, propylene glycol, polyethylene glycols, for example lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono- di- and triglycerides, mono- and di-esters of PEG, etc.

In certain embodiments, the primary vehicle or carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In some embodiments, the saline comprises isotonic phosphate-buffered saline. In certain embodiments, neutral buffered saline or saline mixed with serum albumin are further exemplary vehicles. In certain embodiments, pharmaceutical compositions comprise Tris buffer of about pH 7.0-8.5, or acetate buffer of about pH 4.0-5.5, which can further include sorbitol or a suitable substitute therefore. In certain embodiments, a composition comprising a nucleic acid described herein, with or without at least one additional therapeutic agents, can be prepared for storage by mixing the selected composition having the desired degree of purity with optional formulation agents (Remington's Pharmaceutical Sciences, supra) in the form of a lyophilized cake or an aqueous solution. Further, in certain embodiments, a composition comprising a nucleic acid described herein, with or without at least one additional therapeutic agents, can be formulated as a lyophilized form (e.g., a lyophilized powder or crystalline form, a freeze dried form) using appropriate excipients such as sucrose.

In some embodiments a carrier facilitates the incorporation of a compound into cells or tissues. For example dimethyl sulfoxide (DMSO) is a commonly utilized carrier as it facilitates the uptake of many organic compounds into the cells or tissues of an organism. In some embodiments, a pharmaceutical carrier for a composition described herein can be selected from castor oil, ethylene glycol, monobutyl ether, diethylene glycol monoethyl ether, corn oil, dimethyl sulfoxide, ethylene glycol, isopropanol, soybean oil, glycerin, zinc oxide, titanium dioxide, glycerin, butylene glycol, cetyl alcohol, and sodium hyaluronate.

The compounds and compositions used herein can include any suitable buffers, such as for example, sodium citrate buffer and/or sequestering agents, such as an EDTA sequestering agent. Ingredients, such as meglumine, may be added to adjust the pH of a composition or nucleic acid described herein. Nucleic acids and compositions described herein may comprise sodium and/or iodine, such as organically bound iodine. Compositions and compounds used herein may be provided in a container in which the air is replaced by another substance, such as nitrogen.

In certain embodiments, the optimal pharmaceutical composition will be determined by one skilled in the art depending upon, for example, the intended route of administration, delivery format and desired dosage (see e.g., Remington's Pharmaceutical Sciences, supra). In certain embodiments, such compositions may influence the physical state, stability, rate of in vivo release and rate of in vivo clearance of the nucleic acids or polypeptides of the invention.

In some embodiments, compositions described herein are used to prevent and/or treat a disease. In certain embodiments a composition is administered to a subject at risk of acquiring a disease. A composition that is used to prevent disease is often administered to a subject at risk of acquiring a disease. Any suitable method of administering a pharmaceutical composition to a subject can be used to administer a nucleic acid described herein described herein.

The exact formulation and route of administration for a composition for use according to the methods of the invention described herein can be chosen by the individual physician in view of the patient's condition. See, e.g., Fingl et al. 1975, in “The Pharmacological Basis of Therapeutics,” Ch. 1, p. 1; which is incorporated herein by reference in its entirety. Any suitable route of administration can be used for administration of a pharmaceutical composition or nucleic acid described herein. Non-limiting examples of routes of administration include topical or local (e.g., transdermally or cutaneously, (e.g., on the skin or epidermus), in or on the eye, intranasally, transmucosally, in the ear, inside the ear (e.g., behind the ear drum)), enteral (e.g., delivered through the gastrointestinal tract, e.g., orally (e.g., as a tablet, capsule, granule, liquid, emulsification, lozenge, or combination thereof), sublingual, by gastric feeding tube, rectally, and the like), by parenteral administration (e.g., parenterally, e.g., intravenously, intra-arterially, intramuscularly, intraperitoneally, intradermally, subcutaneously, intracavity, intracranially, intra-articular, into a joint space, intracardiac (into the heart), intracavernous injection, intralesional (into a skin lesion), intraosseous infusion (into the bone marrow), intrathecal (into the spinal canal), intrauterine, intravaginal, intravesical infusion, intravitreal), the like or combinations thereof.

In some embodiments a composition herein is provided to a subject. A composition that is provided to a subject is often provided to a subject for self-administration or for administration to a subject by another (e.g., a non-medical professional). For example a composition described herein can be provided as an instruction written by a medical practitioner that authorizes a patient to be provided a composition or treatment described herein (e.g., a prescription). In another example, a composition can be provided to a subject where the subject self-administers a composition orally, intravenously or by way of an inhaler, for example.

Pharmaceutical composition or nucleic acids herein can be formulated to be compatible with a particular route of administration or use. Compositions for parenteral, intradermal, or subcutaneous administration can include a sterile diluent, such as water, saline solution, fixed oils, polyethylene glycols, glycerin, propylene glycol or other synthetic solvents. The preparation may contain one or more preservatives to prevent microorganism growth (e.g., anti-bacterial agents such as benzyl alcohol or methyl parabens; antioxidants such as ascorbic acid or sodium bisulfite; chelating agents such as EDTA; buffers such as acetates, citrates or phosphates and agents for the adjustment of tonicity such as sodium chloride or dextrose). In certain embodiments, a composition herein is substantially free of a chelator (e.g., a zinc chelator, e.g., EDTA or EGTA).

Compositions for injection include sterile aqueous solutions (where water soluble) or dispersions and sterile powders (e.g., sterile lyophilized preparations) for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (e.g., glycerol, propylene glycol, and polyethylene glycol), and suitable mixtures thereof. Fluidity can be maintained, for example, by the use of a coating such as lecithin, or by the use of surfactants. Antibacterial and anti-bacterial agents include, for example, parabens, chlorobutanol, phenol, ascorbic acid and thimerosal. Including an agent that delays absorption, for example, aluminum monostearate and gelatin can prolong absorption of injectable compositions. Polysorbate 20 and polysorbate 80 can be added into the formulation mixture, for example, up to 1%. Other non-limiting additives include histidine HCl, α,α-trehalose dehydrate.

Alternately, one can administer compositions for use according to the methods of the invention in a local rather than systemic manner, for example, via direct application to the skin, mucous membrane or region of interest for treating, including using a depot or sustained release formulation.

In some embodiments, a pharmaceutical composition comprising a nucleic acid described herein can be administered alone. In other embodiments, a pharmaceutical composition comprising a nucleic acid described herein can be administered in combination with one or more additional materials, for example, as two separate compositions or as a single composition where the additional material(s) is (are) mixed or formulated together with the pharmaceutical composition. For example, without being limited thereto, the pharmaceutical composition can be formulated with additional excipients, additional active ingredients, other pharmaceutical compositions, anti-bacterial medications or other nucleic acids.

The pharmaceutical compositions can be manufactured by any suitable manner, including, e.g., by means of conventional mixing, dissolving, granulating, dragee-making, levigating, emulsifying, encapsulating, entrapping or tableting processes.

Pharmaceutical compositions for use in accordance with the invention thus can be formulated in any suitable manner using one or more physiologically acceptable carriers comprising excipients and auxiliaries, which facilitate processing of the active compounds into preparations which can be used pharmaceutically. Proper formulation can depend upon the route of administration chosen. In particular, any suitable formulation, ingredient, excipient, the like or combinations thereof as listed in “Remington's Pharmaceutical Sciences,” Mack Publishing Co., Easton, Pa., 18th edition, 1990 can be used with a composition described herein. The various nucleic acids and compositions described herein, alone or in combination, can be incorporated into or used with the materials described in Remington's. Any suitable techniques, carriers, and excipients can be used, including those understood in the art; e.g., in Remington's Pharmaceutical Sciences, above, all pages of which are incorporated herein by reference in their entirety, including without limitation for all of the types of formulations, methods of making, etc.

In some embodiments, the composition may be formulated, for example, as a topical formulation. The topical formulation may include, for example, a formulation such as a gel, a cream, a lotion, a paste, an ointment, an oil, and a foam. The composition further may include, for example, an absorption emollient.

In some embodiments, at least part of the affected area of the mammal is contacted with the composition on a daily basis, on an as-needed basis, or on a regular interval such as twice daily, three times daily, every other day, etc. The composition can be administered for a period of time ranging from a single as needed administration to administration for 1 day to multiple years, or any value there between, (e.g., 1-90 days, 1-60 days, 1-30 days, etc.). The dosages described herein can be daily dosages or the dosage of an individual administration, for example, even if multiple administrations occur (e.g., 2 sprays into a nostril).

Some embodiments relate to methods of treating or preventing a disease through administration of compositions described herein to the upper respiratory track/bronchi in a mammal in need thereof, for example, by contacting at least part of the upper respiratory tract/bronchi of a mammal with a therapeutically effective amount of a composition as described above or elsewhere herein. The composition can be, for example, formulated as an aerosol formulation, including formulated for use in a nebulizer or an inhaler. The composition further may include other pharmaceutically acceptable components such as a preservative.

In certain embodiments, the amount of a nucleic acid described herein can be any sufficient amount to prevent, treat, reduce the severity of, delay the onset of or alleviate a symptom of a disease as contemplated herein or a specific indication as described herein.

Compositions for use according to the methods of the invention can be, in some embodiments, aerosolized compositions. The aerosolized composition can be formulated such that the composition has increased solubility and/or diffusivity. The composition can comprise a carrier. A carrier can improve the absorption of the composition, change the viscosity of a composition, improve the solubility of the composition, or improve the diffusivity of a composition compared to a pharmaceutical composition that does not comprise a carrier.

Liquid pharmaceutically administrable compositions can, for example, be prepared by dissolving, dispersing, etc. a nucleic acid described herein as defined above and optional pharmaceutical adjuvants in a carrier (e.g., water, saline, aqueous dextrose, glycerol, glycols, ethanol or the like) to form a solution or suspension. Solutions to be aerosolized can be prepared in any suitable form, for example, either as liquid solutions or suspensions, as emulsions, or in solid forms suitable for dissolution or suspension in liquid prior to aerosol production and inhalation.

For administration by inhalation, the compositions described herein can conveniently be delivered in the form of an aerosol (e.g., through liquid nebulization, dry powder dispersion or meter-dose administration). The aerosol can be delivered from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

For aqueous and other non-pressurized liquid systems, a variety of nebulizers (including small volume nebulizers) can be used to aerosolize the formulations. Compressor-driven nebulizers can utilize jet technology and can use compressed air to generate the liquid aerosol. Such devices are commercially available from, for example, Healthdyne Technologies, Inc.; Invacare, Inc.; Mountain Medical Equipment, Inc.; Pari Respiratory, Inc.; Mada Medical, Inc.; Puritan-Bennet; Schuco, Inc., DeVilbiss Health Care, Inc.; and Hospitak, Inc. Ultrasonic nebulizers generally rely on mechanical energy in the form of vibration of a piezoelectric crystal to generate respirable liquid droplets and are commercially available from, for example, Omron Healthcare, Inc. and DeVilbiss Health Care, Inc. Vibrating mesh nebulizers rely upon either piezoelectric or mechanical pulses to generate respirable liquid droplets. Commercial examples of nebulizers that could be used in certain embodiments include RESPIRGARD II®, AERONEB®, AERONEB® PRO, and AERONEB® GO produced by Aerogen; AERX® and AERX ESSENCE™ produced by Aradigm; PORTA-NEB®, FREEWAY FREEDOM™, Sidestream, Ventstream and I-neb produced by Respironics, Inc.; and PARI LC-PLUS®, PARI LC-STAR®, and e-Flow7m produced by PARI, GmbH. By further non-limiting example, U.S. Pat. No. 6,196,219, is hereby incorporated by reference in its entirety.

In some embodiments, the drug solution can be formed prior to use of the nebulizer by a patient. In other embodiments, the drug can be stored in the nebulizer in solid form. In this case, the solution can be mixed upon activation of the nebulizer, such as described in U.S. Pat. No. 6,427,682 and PCT Publication No. WO 03/035030, both of which are hereby incorporated by reference in their entirety. In these nebulizers, the drug, optionally combined with excipients to form a solid composition, can be stored in a separate compartment from a liquid solvent.

Certain embodiments provide pharmaceutical compositions suitable for use in the technology, which include compositions where the active ingredients are contained in an amount effective to achieve its intended purpose. A “therapeutically effective amount” means an amount sufficient to prevent, treat, reduce the severity of, delay the onset of or inhibit a symptom of a disease. The symptom can be a symptom already occurring or expected to occur. Determination of a therapeutically effective amount is well within the capability of those skilled in the art, especially in light of the detailed disclosure provided herein.

The term “an amount sufficient” as used herein refers to the amount or quantity of an active agent (e.g., a nucleic acid described herein, anti-bacterial medication, and/or a combination of these active agents) present in a pharmaceutical composition that is determined high enough to prevent, treat, reduce the severity of, delay the onset of, or inhibit a symptom of a disease and low enough to minimize unwanted adverse reactions. The exact amount of active agents or combination of active agents required will vary from subject to subject, depending on age, general condition of the subject, the severity of the condition being treated, and the particular combination of drugs administered. Thus, it is not always possible to specify an exact universal amount sufficient to prevent or treat a disease for a diverse group of subjects. As is well known, the specific dosage for a given patient under specific conditions and for a specific disease will routinely vary, but determination of the optimal amount in each case can readily be accomplished by simple routine procedures. Thus, an appropriate “an amount sufficient” to prevent or treat a disease in any individual case may be determined by one of ordinary skill in the art using routine experimentation.

In other embodiments, a therapeutically effective amount can describe the amount necessary for a significant quantity of the composition to contact the desired region or tissue where prevention or treatment of a disease is desired.

The nucleic acids and compositions comprising nucleic acids as described herein can be administered at a suitable dose, e.g., at a suitable volume and concentration depending on the route of administration. Within certain embodiments of the invention, dosages of administered nucleic acids can be from 0.01 mg/kg (e.g., per kg body weight of a subject) to 500 mg/kg, 0.1 mg/kg to 500 mg/kg, 0.1 mg/kg to 400 mg/kg, 0.1 mg/kg to 300 mg/kg, 0.1 mg/kg to 200 mg/kg, 0.1 mg/kg to 150 mg/kg, 0.1 mg/kg to 100 mg/kg, 0.1 mg/kg to 75 mg/kg, 0.1 mg/kg to 50 mg/kg, 0.1 mg/kg to 25 mg/kg, 0.1 mg/kg to 10 mg/kg, 0.1 mg/kg to 5 mg/kg or 0.1 mg/kg to 1 mg/kg. In some aspects the amount of a nucleic acid described herein can be about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg. In some embodiments a therapeutically effective amount of a nucleic acid described herein is between about 0.1 mg/kg to 500 mg/kg, or between about 1 mg/kg and about 300 mg/kg. Volumes suitable for intravenous administration are well known.

In some embodiments a nucleic acid described herein or a pharmaceutical composition comprising a nucleic acid described herein that is formulated for topical or external delivery can include higher amounts of a nucleic acid described herein. For example pharmaceutical composition comprising a nucleic acid described herein that is formulated for topical administration may comprise at least 0.1 mg/ml, at least 1 mg/ml, at least 10 mg/ml, at least 100 mg/ml or at least 500 mg/ml of a nucleic acid described herein.

The compositions can, if desired, be presented in a pack or dispenser device, which can contain one or more unit dosage forms containing the active ingredient. The pack can for example comprise metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions comprising a compound of the invention formulated in a compatible pharmaceutical carrier can also be prepared, placed in an appropriate container, and labeled for treatment of an indicated condition.

In some embodiments a nucleic acid described herein comprises one or more distinguishable identifiers. Any suitable distinguishable identifier and/or detectable identifier can be used for a composition or method described herein. In certain embodiments a distinguishable identifier can be directly or indirectly associated with (e.g., bound to) a nucleic acid described herein. For example a distinguishable identifier can be covalently or non-covalently bound to a nucleic acid described herein. In some embodiments a distinguishable identifier is bound to or associated with a nucleic acid described herein and/or a member of binding pair that is covalently or non-covalently bound to a nucleic acid described herein. In some embodiments a distinguishable identifier is reversibly associated with a nucleic acid described herein. In certain embodiments a distinguishable identifier that is reversibly associated with a nucleic acid described herein can be removed from a nucleic acid described herein using a suitable method (e.g., by increasing salt concentration, denaturing, washing, adding a suitable solvent and/or salt, adding a suitable competitor, and/or by heating).

In some embodiments a distinguishable identifier is a label. In some embodiments a nucleic acid described herein comprises a detectable label, non-limiting examples of which include a radiolabel (e.g., an isotope), a metallic label, a fluorescent label, a chromophore, a chemiluminescent label, an electrochemiluminescent label (e.g., Origen™), a phosphorescent label, a quencher (e.g., a fluorophore quencher), a fluorescence resonance energy transfer (FRET) pair (e.g., donor and acceptor), a dye, a protein (e.g., an enzyme (e.g., alkaline phosphatase and horseradish peroxidase), an antibody, an antigen or part thereof, a linker, a member of a binding pair), an enzyme substrate, a small molecule (e.g., biotin, avidin), a mass tag, quantum dots, nanoparticles, the like or combinations thereof Δny suitable fluorophore or light emitting material can be used as a label. A light emitting label can be detected and/or quantitated by a variety of suitable techniques such as, for example, flow cytometry, gel electrophoresis, protein-chip analysis (e.g., any chip methodology), microarray, mass spectrometry, cytofluorimetric analysis, fluorescence microscopy, confocal laser scanning microscopy, laser scanning cytometry, the like and combinations thereof.

### Binding Pairs

In some embodiments a nucleic acid, or composition described herein comprises one or more binding pairs. In some embodiments a binding pair comprises at least two members (e.g., molecules) that bind non-covalently to (e.g., associate with) each other. Members of a binding pair often bind specifically to each other. Members of a binding pair often bind reversibly to each other, for example where the association of two members of a binding pair can be dissociated by a suitable method. Any suitable binding pair, or members thereof, can be utilized for a composition or method described herein. Non-limiting examples of a binding pair includes antibody/antigen, antibody/antibody, antibody/antibody fragment, antibody/antibody receptor, antibody/protein A or protein G, hapten/anti-hapten, sulfhydryl/maleimide, sulfhydryl/haloacetyl derivative, amine/isotriocyanate, amine/succinimidyl ester, amine/sulfonyl halides, biotin/avidin, biotin/streptavidin, folic acid/folate binding protein, receptor/ligand, vitamin B12/intrinsic factor, analogues thereof, derivatives thereof, binding portions thereof, the like or combinations thereof. Non-limiting examples of a binding pair member include an antibody, antibody fragment, reduced antibody, chemically modified antibody, antibody receptor, an antigen, hapten, anti-hapten, a peptide, protein, nucleic acid (e.g., double-stranded DNA (dsDNA), single-stranded DNA (ssDNA), or RNA), a nucleotide, a nucleotide analog or derivative (e.g., bromodeoxyuridine (BrdU)), an alkyl moiety (e.g., methyl moiety on methylated DNA or methylated histone), an alkanoyl moiety (e.g., an acetyl group of an acetylated protein (e.g., an acetylated histone)), an alkanoic acid or alkanoate moiety (e.g., a fatty acid), a glyceryl moiety (e.g., a lipid), a phosphoryl moiety, a glycosyl moiety, a ubiquitin moiety, lectin, aptamer, receptor, ligand, metal ion, avidin, neutravidin, biotin, B12, intrinsic factor, analogues thereof, derivatives thereof, binding portions thereof, the like or combinations thereof. In some embodiments, a member of a binding pair comprises a distinguishable identifier.

In some embodiments the nucleic acids, compositions, formulations, combination products and materials described herein can be included as part of kits, which kits can include one or more of pharmaceutical compositions, nucleic acids, and formulations of the same, combination drugs and products and other materials described herein. In some embodiments the products, compositions, kits, formulations, etc. can come in an amount, package, product format with enough medication to treat a patient for 1 day to 1 year, 1 day to 180 days, 1 day to 120 days, 1 day to 90 days, 1 day to 60 days, 1 day to 30 days, or any day or number of days there between, 1-4 hours, 1-12 hours, or 1-24 hours.

The invention provides kits including pharmaceutical compositions of the invention, combination compositions and pharmaceutical formulations thereof, packaged into suitable packaging material. A kit optionally includes a label or packaging insert including a description of the components or instructions for use in vitro, in vivo, or ex vivo, of the components therein. Exemplary instructions include instructions for a treatment protocol or therapeutic regimen.

A kit can contain a collection of such components, e.g., two or more conjugates alone, or in combination with another therapeutically useful composition (e.g., an anti-proliferative or immune-enhancing drug). The term “packaging material” refers to a physical structure housing the components of the kit. The packaging material can maintain the components sterilely, and can be made of material commonly used for such purposes (e.g., paper, corrugated fiber, glass, plastic, foil, ampules, vials, tubes, etc.).

Kits can include labels or inserts. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, optical disk such as CD- or DVD-ROM/RAM, DVD, MP3, magnetic tape, or an electrical storage media such as RAM and ROM or hybrids of these such as magnetic/optical storage media, FLASH media or memory type cards.

Labels or inserts can include identifying information of one or more components therein, dose amounts, clinical pharmacology of the active ingredient(s) including mechanism of action, pharmacokinetics (PK) and pharmacodynamics (PD). Labels or inserts can include information identifying manufacturer information, lot numbers, manufacturer location and date.

Labels or inserts can include information on a condition, disorder, disease or symptom for which a kit component may be used. Labels or inserts can include instructions for the clinician or for a subject for using one or more of the kit components in a method, treatment protocol or therapeutic regimen. Instructions can include dosage amounts, frequency or duration, and instructions for practicing any of the methods, treatment protocols or therapeutic regimes set forth herein. Kits of the invention therefore can additionally include labels or instructions for practicing any of the methods and uses of the invention described herein.

Labels or inserts can include information on any benefit that a component may provide, such as a prophylactic or therapeutic benefit. Labels or inserts can include information on potential adverse side effects, such as warnings to the subject or clinician regarding situations where it would not be appropriate to use a particular composition. Adverse side effects could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Kits can additionally include other components. Each component of the kit can be enclosed within an individual container and all of the various containers can be within a single package. Invention kits can be designed for cold storage. Invention kits can further be designed to contain host cells expressing nucleic acids, (e.g., nucleic acids encoding a polypeptide). The cells in the kit can be maintained under appropriate storage conditions until the cells are ready to be used. For example, a kit including one or more cells can contain appropriate cell storage medium so that the cells can be thawed and grown.

Such kits can take any suitable form. For example, a kit can comprise or consist of a stick test, including necessary reagents to perform the method of the invention and to produce, for example, a colorimetric result which can be compared against a color chart or standard curve. Such kits can also comprise, e.g., a buffering agent, a preservative, or a protein stabilizing agent. The kit can also comprise components necessary for detecting a nucleic acid described herein. A kit can also contain a control sample and/or a series of control samples (e.g., controls containing known amounts of one or more nucleic acids or polypeptides) which can be assayed and compared to the test sample contained. In some embodiments, each component of the kit is usually enclosed within an individual container, and all of the various containers are within a single package, along with instructions.

### EXAMPLES

### Example 1—A Modified Argonaute for DNA-Guided Genome Editing in Mammalian Cells

The ability to introduce a nucleic acid (e.g., a gene, heterologous DNA or modified nucleic acid) into a genome of an organism at a specific targeted locus is a powerful tool for therapeutic and research purposes. Zinc finger nucleases (ZFNs), transcription activator-like effector nucleases (TALENs) and clustered regularly interspaced short palindromic repeats (CRISPR)-associated (Cas) nucleases have been used successfully and efficiently by many laboratories for certain genome editing applications. More recently, the RNA-guided endonucleases such as Cas9 and Cpf1 have gained more tractions because of their relatively ease of manipulation. The user-friendly CRISPR-Cas9 is very efficient in making mutations via non-homologous end joining (NHEJ) in human cancer cell lines such as 293T cells. It can also mediate homologous recombination (HR), but at a much low efficiency (2-5%) in 293T cells and even lower in other biologically relevant cells such as human induced pluripotent stem cells (iPSCs).

To validate whether the Argonaute/DNA-guided system could be used as an alternative gene editing tool in mammalian cells, we have carefully evaluated both positive and negative results from published reports (Gao, et al. 2016; Burgess et al 2016). We reasoned that it is plausible for NgAgo to mediate gene editing in human cells, but at a very low efficiency to be detectable under the current conditions. Since NgAgo is a product of haloalkaliphilic archaebacterium N. gregoryi SP2, we determined that the codons used in N. gregoryi may be different from the ones used in the translation machinery of human cells (FIG. 1A). Thus, in order to improve the translation of NgAgo in human cells, a modified NgAgo nucleic acid, termed “HuAgo” was (FIG. 1B) was constructed.

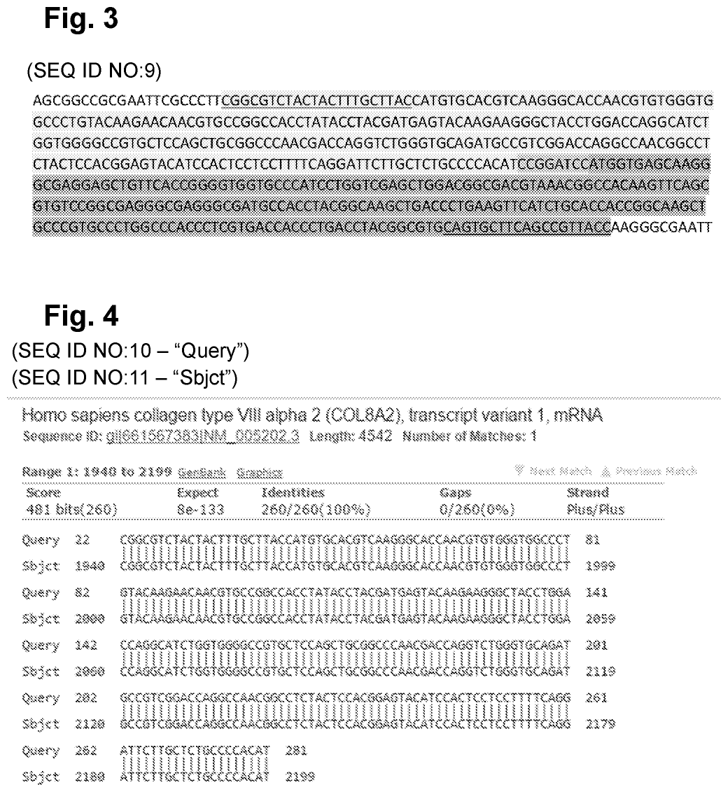

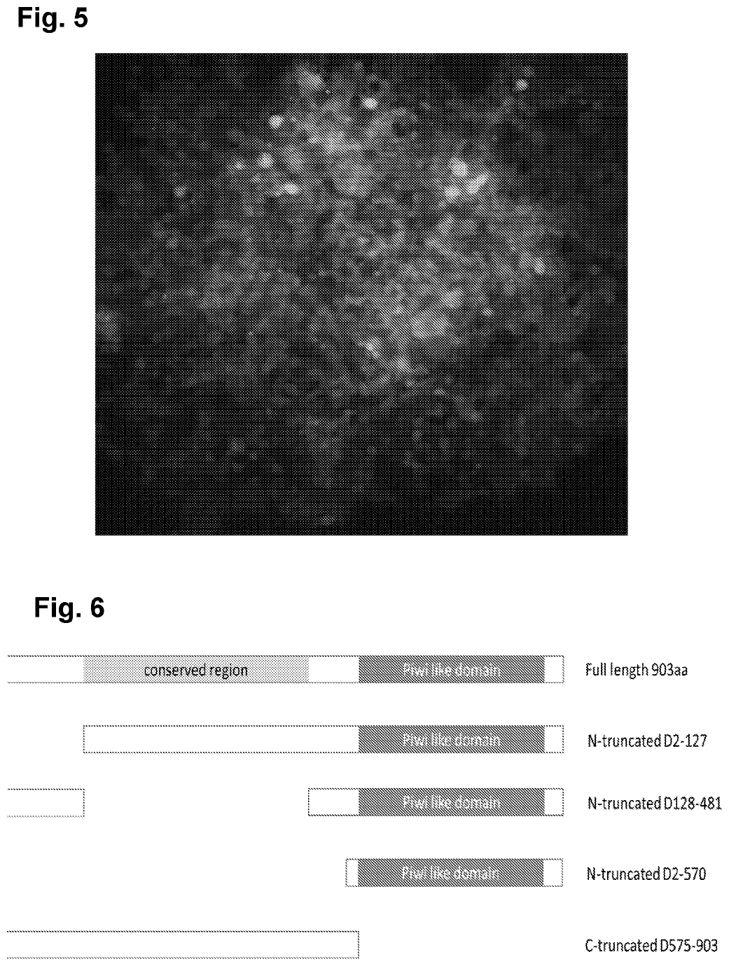

It was determined herein that the length of guide DNA oligo is usually around 20 to 25 nucleotides (nt) and may be a 24-mer in human 293T cells. A 21 nt guide DNA retains about 62% target cleavage activity of a 24 nt length guide DNA. Guide DNA oligonucleotides with either 21 or 24 matched nucleotides were used to target a locus in the last exon of the human COL8A2 gene in HEK293T cells. Both were able to generate DNA breaks when co-transfected with the HuAgo expression vector and a knock-in GFP-puro cassette (i.e., the donor fragment) containing GFP and puromycin resistance coding sequences flanked with a 20-nucleotide length of homology sequence of COL8A2 around the target site at both ends. The knock-in of GFP-puro cassette was targeted to the COL8A2 gene and resulted in the expansion of stable cells that were resistant to puromycin. Sequencing of a PCR product generated from the targeted COL8A2 region confirmed site-specific insertion of GFP and the puromycin resistance coding sequences into the last exon of the human COL8A2 gene. Moreover, these stable cells were confirmed to express GFP (FIG. 3).

In an effort to define a potential motif that is important for the activity of our HuAgo/guide DNA system in mammalian cells, we performed a search using Domain Enhanced Lookup Time Accelerated BLAST (DELTA-BLAST) against the US National Center for Biotechnology Information (NCBI) non-redundant protein sequence database using the amino acid sequence of HuAgo and NgAgo. A conserved Piwi-like domain of 313 amino acids shared by Piwi superfamily was identified. The Piwi-like sequence was located in the C terminal region of HuAgo protein. Between the residue 127 and 482, multiple short stretches of conserved sequences were also found, but they are associated with any known motif. To test whether the Piwi-like domain and the other conserved sequences are indispensable for HuAgo function in the cells, we have made several truncated version of HuAgo constructs as shown in FIG. 4. The comparative analysis of activities of these truncated proteins vs the full length protein is ongoing.

### Example 2

Materials and Methods

Constructs of Argonaute expression cassette. The full length and truncated versions of Human codon-optimized NgAgo sequence (HuAgo) were synthesized and fused to the P2A-YFP cDNA cassette. The expression of fusion open reading frame was driven by EF1alph promoter in a mammalian expression vector (SynBio Tech, New Jersey).

Preparation of EGFP-2A-Puro knock-in donor fragments. A 20-bp nucleic acid sequence homologous to a portion of the target site (FIG. 2, labeled “20 bp”) and a 21-bp nucleic acid homologous to a portion of the target site (FIG. 2, labeled “21 bp”) were added to flank 5′ and 3′ ends of EGFP-2A-Puro cassette respectively by using synthetic oligo primers and PCR to create the donor fragment sequence (FIG. 2). The 1.4 kb PCR product representing the donor fragment was gel purified.

Oligos and primers. All guided DNA oligonucleotides (oligos), primers containing target specific microhomologous 20-mers and primers used for screening genomic insertions were synthesized by Integrated DNA Technologies (Table 1). 5′-phosphorylation of guided DNA oligo was carried out by using T4 polynucleotide kinase (New England BioLab).

Cell culture and transfection. HEK293T (ATCC CRL-3216) cells were maintained in a DMEM high glucose medium supplemented with 10% fetal bovine serum, 2 mM GlutaMax, 1× non-essential amino acids and 100 U/ml penicillin and 100 ug/ml streptomycin. Cells were seeded into 12-well plates one day before transfection. Cells were transfected at about 60% confluence using Lipofectamine 3000 (Thermo Scientific). Specifically, HEK293 were transfected with 400 ng HuAgo-expressing plasmid, 200 ng of guide-DNA oligo (either guideDNA-COL8A2-p24 or guideDNA-COL8A2-p21, as shown in Table 1) and 250 ng of EGFP-2A-Puro knock-in donor fragment in each well. After 3 days of transfection, cells were either harvested for genomic DNA extraction or treated with puromycin selection to establish a culture of stably integrated inserts. The genomic insertion of donor fragment was identified and confirmed by PCR. The COL8A2 FP2 and GFP RP1 primer pair was used to screen for 5′ junction of knock-in insertion. The Puro FP3 and COL8A2 RP1 primer pair was used to screen for 3′ junction of knock-in insertion.

### Discussion and Perspective

Unlike Cas proteins, which only exist in prokaryotes, Argonaute proteins are conserved through evolution and can be identified in virtually all species. In mammalian cells, endogenous Argonaute proteins are considered to be a major component of the RNA-induced silencing complex (RISC), which mediates RNA interference. Argonaute uses a micro RNA (˜22 nt) as a guide for identifying complementary target mRNA. Interestingly, an evolutionarily related enzyme in prokaryotes, Argonaute of the bacterium Thermus thermophiles (TtAgo), was found to be able to use DNA-guided DNA interference as a defense mechanism to protect its host against foreign DNA. TtAgo bound with 5′-phosphorylated single-stranded DNA guide (13-25 nucleotides in length), effectively cleaved a foreign complementary DNA in vivo. We have utilized gene swamping experiments to demonstrate that evolutionarily conserved proteins, such as homeodomain-containing proteins, are not only conserved in structures, but also act in a similar fashion in vivo.

As shown herein the gene sequence of NgAgo was modified to that of SEQ ID NO:1, which modifications resulted in enhanced expression and/or activity of an Argonaute polypeptide in mammalian cells. The HuAgo generated herein acts as a DNA guided gene editing nuclease in mammalian cells.

HuAgo/DNA-guided gene editing technology has three distinctive advantages over RNA-guided CRISPR/Cas approach:
1) Minimal sequence requirement led to low off-target probability. The single strand guide DNA has no target sequence preference and only requires 5′ phosphorylation and a short length of 24 nucleotides matching specifically to the target site. In contrast, Cas nucleases require a scaffold sequence extension of 76 nucleotides at the 3′ tracrRNA in its sgRNAs and a PAM sequence for DNA binding.
2) Guide DNA molecules are much more versatile and stable than guide RNA molecules. A 24mer DNA oligo is significantly easy to design and cheap to synthesize, and can be precisely dosed.
3) HuAgo protein is much smaller than a Cas nuclease. Its 887 amino acids is about ⅔ the length of Cas9 (1,368 amino acids). The small size of HuAgo makes it ideal for in vivo gene editing by AAV, a very promising system for delivery of a package of nuclease, guideDNA and donor template molecules together to target tissues.

The ability to manipulate any genomic sequence by HuAgo/guideDNA technology may have far-reaching implications and opportunities in developing new treatments for many different human diseases and disorders. Some major applications contemplated herein are:

### Cancer Immunotherapy

Cancer immunotherapy involves or uses components of the immune system such as antibodies and T cells to treat cancer patients. Recently, impressive treatment results have been reported in some cases of lymphoma, leukemia and melanoma with adoptive T-cell immunotherapy, in which autologous T cells are engineered to attack cancer antigen ex vivo and transferred back to the patient. The T-cell immunotherapy could be further enhanced via expressing synthetic receptors known as chimeric antigen receptors, or CARs, and knocking out the endogenous T-cell receptors with engineered nucleases such as HuAgo. In addition, knocking out the human leukocyte antigen (HLA) via gene editing could avoid immune rejection of allogeneic cell therapy.

Another useful application is to increase T-cell effector function and broadly enabling immunotherapy for diverse cancer types by knocking out genes of checkpoint inhibitor pathways such as PD-1 and CTLA-4.

### Antiviral Therapy

Gene editing strategy has been used to knock out CCR5, a coreceptor used for primary HIV infection. Currently, several ongoing clinical trials are evaluating this approach in HIV-positive patients. Early study results provide promising proof-of-principle of a gene-editing approach in humans, which show safe engraftment and survival of CCR5-modified T cells and control of viral load in some patients. The gene-editing platform could also been applied to attack viral genomes of various DNA viruses such as hepatitis B virus, herpes simplex virus, and human papilloma virus. To circumvent the high mutability of viral targets, several DNA guides could be used simultaneously to target multiple critical sites in the viral genome.

### Liver-Targeted Gene Editing

Liver is probably one of the most accessible organs for applying gene-editing technology to treat many different diseases. At one hand, HuAgo/guideDNA could be used to correct mutations caused severe diseases including clotting disorders such hemophilia A and hemophilia B, as well as lysosomal storage disorders such as Fabry disease, Gaucher disease, Pompe disease, von Gierke disease, and Hurler and Hunter syndromes. On the other hand, the disruption of particular genes in the tissue may also have a beneficial effect. For example, reducing PCSK9 activity is correlated with decreased LDL level. In contrast to continuous administration of PCSK9 inhibitors, a liver-targeted PCSK9 knockout or variant substitutions could lead to long-lasting effect of lowering cholesterol levels.

### Blindness Treatment

Another highly accessible organ for applying gene-editing technology is eye. Recent successes in clinical trials for the treatment of Leber Congenital Amaurosis type 2 (LCA2) have raised hope of using gene therapy to treat blindness caused by genetic mutations. LCA is the leading cause of childhood blindness and is caused by mutations in at least 18 different genes. Other autosomal dominant disorders such as forms of primary open angle glaucoma, retinitis pigmentosa and Fuchs endothelial corneal dystrophy, could potentially be treated by targeted editing of mutation sites. More importantly, the proven safety of AAV delivery in eyes and the compact size of HuAgo/guideDNA system make it a particularly attractive gene-editing strategy in clinical settings.

Broadly speaking, with the advent of the HuAgo/guideDNA technology, manipulating the genome of plants and animals, in particularly, human pluripotent stem cells will become an easy task, allowing modifying their genome with superior precision, speed, and throughput. It will revolutionize scientific communities for both developing models to understand biological processes and strategies for improving wellness of living organisms.

In certain aspects, the single stranded oligodeoxynucleotide-mediated knock-in protocol described herein can be applied to any target site with any donor vector without the need to construct homology arms, thus simplifying genome engineering in living organisms.

In some embodiments, the nucleic acids, compositions and methods described herein are useful for additional application, non-limiting examples of which include single and multiplex gene knockouts, conditional gene knockouts, generation of knock-in alleles, introduction of small as well as large genetic modifications, generation of large deletions and chromosome engineering, genome-wide screens, transcriptional regulation, genetic modifications of mitochondrial DNA and target Mitochondrial diseases, and the like.

TABLE 1
Oligos and primers used in this study

| Oligos or | | Length | |
|---|---|---|---|
| Primers | Sequences | (nt) | Use |
| guideDNA- | SEQ ID NO: 16 | 24 | guide DNA |
| COL8A2- | 5′-GCCCCACATAACCCGCGGGGGGGG-3′ | | |
| p24 | | | |
| guideDNA- | SEQ ID NO: 17 | 25 | guide DNA |
| Col8A2- | 5′-CACCggaccccccccgcgggttatg-3′ | | |
| p21 | | | |
| COL-GFP- | SEQ ID NO: 18 | 59 | 5′ flanking |
| F59 | 5′ATCCACTCCTCCTTTTCAGGATTCTTGCTC | | PCR insert |
| | TGCCCCACATATGGTGAGCAAGGGCGAGG 3′ | | primer |
| COL-PURO- | SEQ ID NO: 19 | 59 | 3′ flanking |
| R59 | 5′AACTAAAGGGGAGGAGGCCAGGGCAGCAGGA | | PCR insert |
| | CCCCCCCCGCGGGTTTCAGGCACCGGGCT 3′ | | primer |
| Col8A2 | SEQ ID NO: 20 | 22 | 5′ genomic |
| FP1 | CGGCGTCTACTACTTTGCTTAC | | junctional |
| | | | screen |
| GFP RP1 | SEQ ID NO: 21 | 19 | 5′ genomic |
| | GGTAACGGCTGAAGCACTG | | junctional |
| | | | screen |
| COL8A2 | SEQ ID NO: 22 | 21 | 3′ genomic |
| RP1 | AGCCTGCATGCAGGGAGAAAG | | junctional |
| | | | screen |
| Puro FP3 | SEQ ID NO: 23 | 21 | 3′ genomic |
| | GAGCTGCAAGAACTCTTCCTC | | junctional |
| | | | screen |

SEQ ID NO: 1
CCACCGTGATCGACCTGGACTCCACCACCACCGCCGACGAGCTGACCTC
CGGCCACACCTACGACATCTCCGTGACCCTGACCGGCGTGTACGACAAC
ACCGACGAGCAGCACCCCCGGATGTCCCTGGCCTTCGAGCAGGACAACG
GCGAGCGGCGGTACATCACCCTGTGGAAGAACACCACCCCCAAGGACGT
GTTCACCTACGACTACGCCACCGGCTCCACCTACATCTTCACCAACATC
GACTACGAGGTGAAGGACGGCTACGAGAACCTGACCGCCACCTACCAGA
CCACCGTGGAGAACGCCACCGCCCAGGAGGTGGGCACCACCGACGAGGA
CGAGACCTTCGCCGGCGGCGAGCCCCTGGACCACCACCTGGACGACGCC
CTGAACGAGACCCCCGACGACGCCGAGACCGAGTCCGACTCCGGCCACG
TGATGACCTCCTTCGCCTCCCGGGACCAGCTGCCCGAGTGGACCCTGCA
CACCTACACCCTGACCGCCACCGACGGCGCCAAGACCGACACCGAGTAC
GCCCGGCGGACCCTGGCCTACACCGTGCGGCAGGAGCTGTACACCGACC
ACGACGCCGCCCCCGTGGCCACCGACGGCCTGATGCTGCTGACCCCCGA
GCCCCTGGGCGAGACCCCCCTGGACCTGGACTGCGGCGTGCGGGTGGAG
GCCGACGAGACCCGGACCCTGGACTACACCACCGCCAAGGACCGGCTGC
TGGCCCGGGAGCTGGTGGAGGAGGGCCTGAAGCGGTCCCTGTGGGACGA
CTACCTGGTGCGGGGCATCGACGAGGTGCTGTCCAAGGAGCCCGTGCTG
ACCTGCGACGAGTTCGACCTGCACGAGCGGTACGACCTGTCCGTGGAGG
TGGGCCACTCCGGCCGGGCCTACCTGCACATCAACTTCCGGCACCGGTT
CGTGCCCAAGCTGACCCTGGCCGACATCGACGACGACAACATCTACCCC
GGCCTGCGGGTGAAGACCACCTACCGGCCCCGGCGGGGCCACATCGTGT
GGGGCCTGCGGGACGAGTGCGCCACCGACTCCCTGAACACCCTGGGCAA
CCAGTCCGTGGTGGCCTACCACCGGAACAACCAGACCCCCATCAACACC
GACCTGCTGGACGCCATCGAGGCCGCCGACCGGCGGGTGGTGGAGACCC
GGCGGCAGGGCCACGGCGACGACGCCGTGTCCTTCCCCCAGGAGCTGCT
GGCCGTGGAGCCCAACACCCACCAGATCAAGCAGTTCGCCTCCGACGGC
TTCCACCAGCAGGCCCGGTCCAAGACCCGGCTGTCCGCCTCCCGGTGCT
CCGAGAAGGCCCAGGCCTTCGCCGAGCGGCTGGACCCCGTGCGGCTGAA
CGGCTCCACCGTGGAGTTCTCCTCCGAGTTCTTCACCGGCAACAACGAG
CAGCAGCTGCGGCTGCTGTACGAGAACGGCGAGTCCGTGCTGACCTTCC
GGGACGGCGCCCGGGGCGCCCACCCCGACGAGACCTTCTCCAAGGGCAT
CGTGAACCCCCCCGAGTCCTTCGAGGTGGCCGTGGTGCTGCCCGAGCAG
CAGGCCGACACCTGCAAGGCCCAGTGGGACACCATGGCCGACCTGCTGA
ACCAGGCCGGCGCCCCCCCCACCCGGTCCGAGACCGTGCAGTACGACGC
CTTCTCCTCCCCCGAGTCCATCTCCCTGAACGTGGCCGGCGCCATCGAC
CCCTCCGAGGTGGACGCCGCCTTCGTGGTGCTGCCCCCCGACCAGGAGG
GCTTCGCCGACCTGGCCTCCCCCACCGAGACCTACGACGAGCTGAAGAA
GGCCCTGGCCAACATGGGCATCTACTCCCAGATGGCCTACTTCGACCGG
TTCCGGGACGCCAAGATCTTCTACACCCGGAACGTGGCCCTGGGCCTGC
TGGCCGCCGCCGGCGGCGTGGCCTTCACCACCGAGCACGCCATGCCCGG
CGACGCCGACATGTTCATCGGCATCGACGTGTCCCGGTCCTACCCCGAG
GACGGCGCCTCCGGCCAGATCAACATCGCCGCCACCGCCACCGCCGTGT
ACAAGGACGGCACCATCCTGGGCCACTCCTCCACCCGGCCCCAGCTGGG
CGAGAAGCTGCAGTCCACCGACGTGCGGGACATCATGAAGAACGCCATC
CTGGGCTACCAGCAGGTGACCGGCGAGTCCCCCACCCACATCGTGATCC
ACCGGGACGGCTTCATGAACGAGGACCTGGACCCCGCCACCGAGTTCCT
GAACGAGCAGGGCGTGGAGTACGACATCGTGGAGATCCGGAAGCAGCCC
CAGACCCGGCTGCTGGCCGTGTCCGACGTGCAGTACGACACCCCCGTGA
AGTCCATCGCCGCCATCAACCAGAACGAGCCCCGGGCCACCGTGGCCAC
CTTCGGCGCCCCCGAGTACCTGGCCACCCGGGACGGCGGCGGCCTGCCC
CGGCCCATCCAGATCGAGCGGGTGGCCGGCGAGACCGACATCGAGACCC
TGACCCGGCAGGTGTACCTGCTGTCCCAGTCCCACATCCAGGTGCACAA
CTCCACCGCCCGGCTGCCCATCACCACCGCCTACGCCGACCAGGCCTCC
ACCCACGCCACCAAGGGCTACCTGGTGCAGACCGGCGCCTTCGAGTCCA
ACGTGGGCTTCCTGTCTAGA

### Example 3 Base Modification of Endogenous Human CD274 Gene with HuAgo and Guide DNA

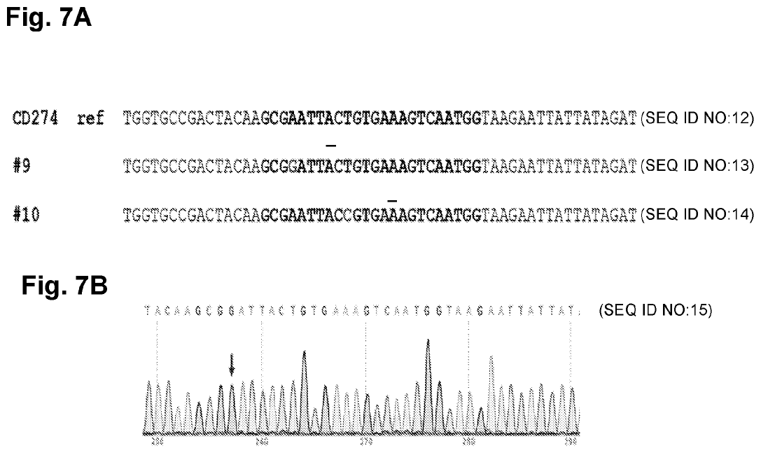

To test if HuAgo could be used to modify an endogenous human gene such as CD274 or PDL-1 for potential application of cancer therapy, a guide DNA oligo GD5 (5′-pGCGAATTACTGTGAAAGTCAATGG-3′) complimentary to a region of Exon 3 of CD274 gene was used. HEK293T cells were seeded into 12-well plates one day before transfection. The HuAgo expression plasmid as described above (Example 1) and GD5 were co-transfected by using Lipofectamine 3000 (ThermoFisher Scientific). A donor fragment was not used in these experiments. The genomic DNA of transfected cells was harvested at 72 hours post-transfection. A 461 bp amplicon containing the exon 3 target site was generated by PCR with a specific CD274_461F/R primer pair (461F primer: 5′-CCT GGC TGC ACT AAT TGT CTA T-3; 461R primer: 5′-CTG TGT TGT TTG TTC TGG ATT TC-3′). The amplicon was inserted into pCR4-TOPO cloning vector (ThermoFisher Scientific) for sequencing. Among 18 amplicon colonies, whose plasmid DNAs were miniprepared and sequenced with T7 universal primer (Eton Biosciences), clones #9 and #10 showed A to G and T to C base changes at +4 and +10 position of the GD5 target site respectively (FIG. 7). The result suggests that the HuAgo/guide molecular system can be used to alter single bases at a targeted genomic location. This is more desirable than the conventional CRISPR/Cas based method, which is a relatively unpredictable and blunt form of molecular scissors that often remove a sizable section of genomic DNA.

### Example 4 Embodiments

A1. A synthetic nucleic acid comprising a first nucleic acid sequence having greater than 82% identity to the nucleic acid sequence of SEQ ID NO:1.
A2. The synthetic nucleic acid of embodiment A1, wherein the first nucleic acid sequence has at least 85% identity to the nucleic acid sequence of SEQ ID NO:1.
A3. The synthetic nucleic acid of embodiment A1, wherein the first nucleic acid sequence has at least 90% identity to the nucleic acid sequence of SEQ ID NO:1.
A4. The synthetic nucleic acid of embodiment A1, wherein the first nucleic acid sequence comprises the nucleic acid sequence of SEQ ID NO:1.
B1. A synthetic nucleic acid comprising a first nucleic acid comprising at least 30, at least 100, at least 300, at least 500, at least 1000, or at least 2000 contiguous nucleotides of the nucleic acid sequence of SEQ ID NO:1.
B2. The synthetic nucleic acid of embodiment B1, wherein the first nucleic acid sequence comprises at least 500 contiguous nucleotides of the nucleic acid sequence of SEQ ID NO:1.
B3. The synthetic nucleic acid of embodiment B1, wherein the first nucleic acid sequence comprises at least 750 contiguous nucleotides of the nucleic acid sequence of SEQ ID NO:1.
B3. The synthetic nucleic acid of embodiment B1, wherein the first nucleic acid sequence comprises at least 1000 contiguous nucleotides of the nucleic acid sequence of SEQ ID NO:1.
C1. A synthetic nucleic acid comprising a first nucleic acid sequence consisting of at least 30, at least 40, at least 100, at least 300, at least 500, at least 1000, or at least 2000 nucleotides in length, wherein the first nucleic acid has at least 80%, at least 90% or at least 95% identity to a portion of the nucleic acid sequence of SEQ ID NO:1, SEQ ID NO:4 or SEQ ID NO:5.
C2. The synthetic nucleic acid of embodiment C1, wherein the first nucleic acid sequence is at least 1000 nucleotides in length.
C3. The synthetic nucleic acid of embodiment C1 or C2, wherein the first nucleic acid has at least 90% identity to a portion of the nucleic acid sequence of SEQ ID NO:1, SEQ ID NO:4 or SEQ ID NO:5.
C4. The synthetic nucleic acid of any one of embodiments C1 or C3, wherein the first nucleic acid has comprises the nucleic acid sequence of SEQ ID NO:1, SEQ ID NO:4 or SEQ ID NO:5.
D1. The synthetic nucleic acid of any one of embodiments A1 to A4, B1 to B3, or C1 to C4, further comprising a promoter.
D2. The synthetic nucleic acid of embodiment D1, further comprising a nuclear localization signal (NLS) sequence.
E1. A composition comprising the synthetic nucleic acid of any one of embodiments A1 to D2.
E2. The composition of embodiment E1, wherein the composition is a pharmaceutical composition.
E3. The composition of embodiment E1 or E2, further comprising a pharmaceutical acceptable excipient, diluent, additive or carrier.
E4. The composition of any one of embodiments E1 to E2, further comprising one or more guide oligonucleotides.
E5. The composition of any one of embodiments E1 to E4, further comprising one or more donor sequences.
F1. A kit comprising the synthetic nucleic acid of any one of embodiments A1 to D2, or the composition of any one of embodiments E1 to E5.
G1. A method of editing a genome of a cell comprising:

a) providing a cell comprising a genome;

b) introducing into the cell
(i) the synthetic nucleic acid of any one of embodiments A1 to D2, wherein the synthetic nucleic acid is configured to express a synthetic Argonaute polypeptide,
(ii) a first guide oligonucleotide consisting of 18 to 27 nucleotides in length, and comprising a nucleotide sequence that is at least 85% identical to a nucleic acid sequence in the genome;
(iii) a second guide oligonucleotide consisting of 18 to 27 nucleotides in length, and comprising a nucleotide sequence that is at least 85% identical to a nucleic acid sequence in the genome; and
(iv) a desired nucleic acid comprising a nucleic acid sequence of the first guide oligonucleotide and a nucleic acid sequence of the second oligonucleotide guide,
wherein the desired nucleic acid is integrated into the genome of the cell.
   G2. The method of embodiment G1, wherein the cell is a mammalian cell.
   G3. The method of embodiment G1 or G2, wherein the synthetic nucleic acid encodes a synthetic Argonaute polypeptide.
   G4. The method of any one of embodiments G1 to G3, wherein the synthetic Argonaute polypeptide comprises nuclease activity.
   G5. The method of any one of embodiments G1 to G4, wherein the first guide oligonucleotide and the second guide oligonucleotide have different sequences.
   G6. The method of any one of embodiments G1 to G4, wherein the first guide oligonucleotide and the second guide oligonucleotide are the same.
   G7. The method of any one of embodiments G1 to G6, wherein the first guide oligonucleotide or the second guide oligonucleotide consists of 20 to 25 nucleotides in length.
   G8. The method of any one of embodiments G1 to G7, wherein the first guide oligonucleotide or the second guide oligonucleotide comprises a nucleotide sequence that is at least 95% identical to a nucleic acid sequence in the genome.
   G9. The method of any one of embodiments G1 to G8, wherein the desired nucleic acid comprises a heterologous nucleic acid sequence that is flanked by the nucleic acid sequence of the first guide oligonucleotide and the nucleic acid sequence of the second oligonucleotide guide.
   G10. The method of any one of embodiments G1 to G9, wherein the heterologous nucleic acid sequence is configured to express a heterologous polypeptide.
   G11. The method of any one of embodiments G1 to G10, wherein the heterologous polypeptide is a chimeric antigen receptor (CAR).
   G12. The method of any one of embodiments G1 to G11, wherein the first guide oligonucleotide and/or second guide oligonucleotide is phosphorylated at the 5′-hydroxyl.
   H1. A synthetic nucleic acid comprising a first nucleic acid sequence configured to express an Argonaute polypeptide or functional fragment thereof, wherein the first nucleic acid consists of at least 300 contiguous nucleotides having at least 82% identity to the nucleic acid sequence of SEQ ID NO:1, or portion thereof, and the first nucleic acid comprises a coding region that encodes the Argonaute polypeptide or functional fragment thereof.
   H2. The synthetic nucleic acid of embodiment H1, wherein the first nucleic acid further comprises a promoter operably linked to the coding region.
   H3. The synthetic nucleic acid of embodiment H1, wherein the first nucleic acid comprises a nuclear localization signal (NLS) sequence.
   H4. A composition comprising the synthetic nucleic acid of any one of embodiments H1 to H3.
   H5. The composition of embodiment H4, further comprising one or more guide oligonucleotides wherein each of the one or more guide oligonucleotides are 18 to 30 nucleotides in length and each of the one or more guide oligonucleotides consists of a nucleic acid sequence that is greater than 90% identical to a nucleic acid sequence in the genome of a mammal.
   H6. The composition of embodiment H4 or H5, wherein the composition comprises a donor nucleic acid comprising a desired nucleic acid that is flanked by a nucleic acid sequence of the one or more guide oligonucleotides.
   H7. The composition of any one of embodiments H4 to H6, wherein the composition is a pharmaceutical composition comprising a pharmaceutical acceptable excipient, diluent, additive or carrier.
   H8. A kit comprising the synthetic nucleic acid of any one of embodiments H1 to H3, or the composition of any one of embodiments H4 to H7.
   H9. A method of editing a genome of an organism or cell comprising:
a) providing a cell or organism comprising a genome;
b) contacting the cell or organism with (i) the synthetic nucleic acid of any one of embodiments H1 to H3, (ii) a first guide oligonucleotide consisting of 18 to 30 nucleotides in length, and a nucleotide sequence that is at least 90% identical to a nucleic acid sequence in the genome; (iii) a second guide oligonucleotide consisting of 18 to 30 nucleotides in length, and a nucleotide sequence that is at least 90% identical to a nucleic acid sequence in the genome; and (iv) a donor nucleic acid comprising a desired nucleic acid sequence, a nucleic acid sequence of the first guide oligonucleotide and a nucleic acid sequence of the second oligonucleotide guide, wherein the synthetic nucleic acid, the first oligonucleotide guide, the second guide oligonucleotide and the donor nucleic acid are introduced into the cell and the desired nucleic acid is integrated into the genome of the cell.
   H9.1. A method of modifying a target sequence in a genome of a cell comprising:
a) providing a cell comprising a genome;
b) contacting the cell with (i) the synthetic nucleic acid of any one of embodiments A1 to D2, and H1 to H3, and (ii) a guide oligonucleotide consisting of 18 to 30 nucleotides in length, and having at least 90% identical to a nucleic acid sequence in the genome.
   H9.2. The method of embodiment H9.1, wherein the contacting of (b) further comprises contacting the cell with (iii) a donor nucleic acid comprising (e.g., from a 5′ to a 3′ direction) a 5′-flanking sequence, a desired nucleic acid sequence, and a 3′ flanking sequence.
   H9.3. The method of embodiment H9.2, wherein the 5′-flanking sequence and the 3′-flanking sequence each comprise at least 10 consecutive nucleotides of the target sequence and at least 10 consecutive nucleotides of the guide oligonucleotide.
   H9.4. The method of any one of embodiments H9 to H9.3, wherein the contacting of (b) comprises introducing the synthetic nucleic acid, the guide oligonucleotide and/or the donor nucleic acid into the cell.
   H9.5. The method of any one of embodiments H9 to H9.4, wherein the synthetic nucleic acid encodes an Argonaute polypeptide, or functional fragment thereof.
   H9.6. The method of any one of embodiments H9 to H9.5, wherein the Argonaute polypeptide or functional fragment thereof is expressed in the cell.
   H9.7. The method of any one of embodiments H9 to H9.6, wherein desired nucleic acid is at least 1, at least 10, at least 100 or at least 1000 nucleotides in length.
   H9.8. The method of any one of embodiments H9 to H9.7, wherein the desired nucleic acid is integrated into the genome of the cell within the target sequence.
   H9.9. The method of any one of embodiments H9 to H9.6, wherein the editing or modifying comprises inserting, deleting or replacing one or more nucleotides within the target sequence.
   H10. The method of any one of embodiments H9 to H9.9, wherein the cell is a mammalian cell or a human cell.
   H11. The method of any one of embodiments H9 or H10, wherein the first guide oligonucleotide and/or the second guide oligonucleotide have different sequences.
   H12. The method of any one of embodiments H9 or H10, wherein the first guide oligonucleotide and/or the second guide oligonucleotide are the same.
   H13. The method of any one of embodiments H9 to H12, wherein the desired nucleic acid comprises a human gene or portion thereof.
   G1. A synthetic nucleic acid comprising a first nucleic acid having 80% to 100% identity to the nucleic acid sequence of SEQ ID NO:1, SEQ ID NO:3, SEQ ID NO:4 or SEQ ID NO:5, or a portion thereof.
   G2. The synthetic nucleic acid of embodiment G1, wherein the first nucleic acid comprises the nucleic acid sequence of SEQ ID NO:1, SEQ ID NO:3, SEQ ID NO:4 or SEQ ID NO:5, or a portion thereof.
   G3. The synthetic nucleic acid of embodiment G1 or G2, wherein the first nucleic acid is at least 300, at least 500, at least 1000, at least 2000, or at least 2500 in length.
   G4. The synthetic nucleic acid of embodiment G1 or G2, wherein the first nucleic acid encodes a functional Argonaute polypeptide or functional fragment thereof.
   G5. A composition comprising the synthetic nucleic acid of any one of embodiments G1 to G4, and a guide sequence that is 18-30 nucleotides in length.
   G6. The composition of embodiment G5, further comprising a donor sequence comprising (i) a 5′-flanking sequence, (ii) a desired sequence and (iii) a 3′-flanking sequence, wherein each of the 5′-flanking sequence and the 3′-flanking sequence comprise at least 10 nucleotides that are identical to the guide sequence.
   G7. The composition of embodiment G5 or G6, wherein the guide sequence is at least 90% identical to a target nucleic acid sequence in the genome of a human cell.

The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It will be understood by those of skill in the art that numerous and various modifications can be made without departing from the spirit of the technology. Therefore, it should be clearly understood that the forms of the technology are illustrative only and are not intended to limit the scope of the technology.

All references cited herein are hereby incorporated by reference in their entirety.

1. A synthetic nucleic acid comprising a first nucleic acid sequence comprising 1000 contiguous nucleotides having at least 82% identity to the nucleic acid sequence of SEQ ID NO:1, or portion thereof; wherein the first nucleic acid sequence encodes an Argonaute polypeptide, or functional fragment thereof.

2. The synthetic nucleic acid of claim 1, wherein the first nucleic acid comprises the nucleic acid sequence of SEQ ID NO:1.

3. The synthetic nucleic acid of claim 1, wherein the first nucleic acid comprising a coding region that encodes an Argonaute polypeptide, or functional fragment thereof.

4. The synthetic nucleic acid of claim 3, further comprising a promoter operably linked to the coding region.

5. The synthetic nucleic acid of claim 1, further comprising a nuclear localization signal (NLS) sequence.

6. A composition comprising the synthetic nucleic acid of claim 1.

7. The composition of claim 6, further comprising a guide oligonucleotide that is 18 to 30 nucleotides in length, wherein the guide oligonucleotide is at least 90% identical to a target sequence located in the genome of a mammalian cell.

8. (canceled)

9. The composition of claim 7, further comprising a donor nucleic acid comprising (i) a desired nucleic acid sequence, (ii) a 5′-flanking sequence, and (iii) a 3′-flanking sequence, wherein each of the 5′-flanking sequence and the 3′-flanking sequence independently comprise at least 10 consecutive nucleotides that are identical to the guide sequence.

10. The composition of claim 7, wherein the synthetic nucleic acid, the guide oligonucleotide and the donor nucleic acid are separate nucleic acid fragments.

11. The composition of claim 7, wherein the synthetic nucleic acid, the guide oligonucleotide, and the donor nucleic acid are not covalently linked.

12. The composition of claim 6, wherein the composition is a pharmaceutical composition comprising a pharmaceutical acceptable excipient, diluent, additive or carrier.

13. A kit comprising the synthetic nucleic acid of claim 1, or a composition thereof.

14. A method of editing a genome of a cell or an organism comprising:
contacting the cell or the organism with
(i) the synthetic nucleic acid of claim 1, and
(ii) a guide oligonucleotide consisting of 18 to 30 nucleotides in length that is at least 90% identical to a target sequence in the genome.

15. The method of claim 14, further comprising contacting the cell or the organism with,
(iii) a donor nucleic acid comprising,
a desired nucleic acid sequence,
a 5′-flanking sequence, and
a 3′-flanking sequence,
wherein each of the 5′-flanking sequence and the 3′-flanking sequence are located on opposite sides of the desired nucleic acid sequence and independently comprise at least 8 consecutive nucleotides that are identical to a portion of the guide sequence.

16. The method of claim 15, wherein the 5′-flanking sequence and the 3′-flanking sequence are 10 to 50 nucleotides in length.

17. The method of claim 15, wherein each of the 5′-flanking sequence and the 3′-flanking sequence comprise at least 10 nucleotides that are identical to the target sequence.

18. (canceled)

19. (canceled)

20. The method of claim 14, further comprising introducing the synthetic nucleic acid, the guide oligonucleotide, and the donor nucleic acid into the cell.

21. The method of claim 20, wherein the cell is a mammalian cell or a human cell.

22. (canceled)

23. The method of claim 14, wherein the target sequence is modified.

24. The method of claim 23, wherein the modification comprises a deletion, an insertion, replacement of one or more nucleotides, or a combination thereof.

25. (canceled)
